(19) United States
(12) Patent Application Publication
(10) Pub. No.: US 2022/0249062 A1
(43) Pub. Date: Aug. 11, 2022

(54) ULTRASOUND IMAGING APPARATUS AND ULTRASOUND IMAGING METHOD

(71) Applicant: FUJIFILM Healthcare Corporation, Kashiwa-shi (JP)

(72) Inventors: Hiroki TANAKA, Chiba (JP); Takayuki IWASHITA, Chiba (JP); Kazuhiro AMINO, Chiba (JP); Hiroshi KURIBARA, Chiba (JP)

(21) Appl. No.: 17/575,716

(22) Filed: Jan. 14, 2022

(30) Foreign Application Priority Data
Feb. 9, 2021 (JP) 2021-019086

(51) Int. Cl. A61B 8/08; A61B 8/00

(52) U.S. Cl. CPC A61B 8/5207; A61B 8/4494

(57) ABSTRACT

An image having high resolution in the short axis direction is obtained with a simple configuration. A first transmit aperture, and a second transmit aperture whose aperture size in the short axis direction is larger than the first transmit aperture, are sequentially set in a probe where transducers are arranged in each of the long axis direction and the short axis direction, and the first transmission beam and the second transmission beam are transmitted therefrom respectively. These transmissions generate the first received beam signal and the second received beam signal which are weighted in the depth direction and synthesized. In the first region with a shallow depth, the weight of the first received beam signal is increased, whereas in the second region deeper than the first region, the weight of the second received beam signal is increased.

### BACKGROUND OF THE INVENTION

### Field of the Invention

The present invention relates to an ultrasound imaging apparatus.

### Description of the Related Art

Ultrasonic diagnostic apparatuses transmit ultrasonic waves into a subject and receive reflected waves therefrom, through an ultrasound probe, thereby acquiring biometric information of the subject (images within the subject).

Electric pulses are applied from a main unit of the apparatus at different delay times to a plurality of electro-acoustic conversion elements (transducers) in the ultrasound probe. Transmission beams are formed by the plurality of transducers, and the subject is irradiated with the transmission beams. Then, the same ultrasound probe receives the reflected waves from within the subject. Thus received reflected waves are subjected to amplification, delay-and-sum beamforming, quadrature detection, compression processing by a circuit such as a signal processing circuit, and further processing steps including image processing are performed, and then, an image is created. As the ultrasound probe used in this kind of ultrasonic diagnostic apparatus, a 1D array probe and a 2D array probe are particularly known.

The 1D array probe has a structure in which a plurality of transducers is arranged in an array in one direction (hereinafter, referred to as a long axis direction or an azimuth direction). Upon transmission, a delay is given to each time when an electric pulse is inputted in each transducer arranged in the long axis direction of the probe, and this produces a transmission beam that focuses at a desired position in a cross section including the long axis direction and perpendicular to a plane of the transducers. This transmission generates reflected waves from the subject, and an image is created from the reflected waves. In the direction perpendicular to the long axis direction of the 1D array probe (hereinafter, referred to as a short axis direction or an elevation direction), a focal position and an aperture width when transmitting from the 1D array probe are uniquely determined by an acoustic lens or a concave transducer.

The 2D array probe has a configuration in which a plurality of transducers is arranged two-dimensionally in the long axis direction and the short axis direction. The 2D array probe has transceiver circuitry for each transducer and drives each transducer individually, thereby randomly setting the focal position and the aperture width of the transmission beam in three-dimensional space. This allows reduction of depth dependence basically in the azimuth and elevation directions, depending on the aperture widths of the long and short axes. The 2D array probes, however, have not become popular in many ultrasonic diagnostic apparatuses because of increased size/weight, increased control circuit scale, and higher manufacturing costs.

Japanese Unexamined Patent Application Publication No. 2020-65629 (hereinafter, referred to as Patent Document 1) discloses an ultrasonic diagnostic apparatus having a configuration that aims to obtain a good spatial resolution while reducing the acoustic power applied to a living body, upon delivering the transmission beam from 2D array probe, and after delivering a first transmission beam from the first transmit aperture long in the first axial direction, a second transmission beam is delivered to the same position from the second transmit aperture long in the second axial direction. Frame data or volume data obtained by the first transmission beam and the second transmission beam are synthesized respectively at the same position.

There is known a probe with fewer transducers (several or ten pieces or so) in the short-axis direction relative to a common 2D array probe, having a function of changing the dimension size in the short-axis direction by switching operation. It is called a 1.25D array probe. Also known is a probe capable of giving a delay time symmetrically in the short axis direction about the transducer in the center of the short axis direction. This probe is called a 1.5D array probe. In addition, there is also devised a probe that allows for some extent of scanning in the short axis direction of the transmission beam, simultaneously with scanning in the long axis direction thereof, and it is called a 1.75D array probe.

Japanese Patent No. 5921133 (hereinafter, referred to as Patent Document 2) discloses a device having a function of selectively driving a plurality of transducers in the short axis direction by a switch and multiple times of transmission and reception, thereby effectively obtaining 1.5D equivalent images, even though a circuitry scale of the main unit is limited.

### SUMMARY OF THE INVENTION

### Technical Problem

The 1D array probe uniquely determines the focal position and the aperture width in the short axis direction at the time of transmission through the acoustic lens and others. On the short axis plane, therefore, the transmission beam width is small at a certain focal position, but the transmission beam is spread in other portions, causing reduction of resolution in the elevation direction.

The 2D array probe is able to set the focal position at a desired position and to perform scanning, not only in the long axis direction but also in the short axis direction. However, the size and weight of the probe are increased, and a circuit scale for controlling grows.

In the method using the 2D arrays in Patent Document 1, the second transmission beam is transmitted from the second transmit aperture long in the second axial direction, after transmitting the first transmission beam from the first transmit aperture long in the first axial direction, and then frame data or volume data obtained by these transmissions are synthesized. Since this method needs transmission using a plurality of different apertures in the longitudinal direction, in order to generate one image, it takes time to update the image.

An object of the present invention is to provide an ultrasound imaging apparatus capable of obtaining a high-resolution image in the short axis direction, with a simple configuration that does not need independent delaying of electric pulses inputted in each transducer arranged in the short axis direction of the probe.

### Solution to Problem

According to the present invention, there is provided an ultrasound imaging apparatus having a transmitter, a receiver, an image former, and a synthesizer as described below. The transmitter sequentially sets a first transmit aperture and a second transmit aperture, the first transmit aperture having a predetermined size in a short axis direction of a probe and the second transmit aperture having the size in the short axis direction of the probe larger than the first transmit aperture, in the probe where transducers are arranged in each of a long axis direction and the short axis direction of the probe, the transmitter outputting transmission signals to the transducers in each of the first transmit aperture and the second transmit aperture, thereby transmitting a first transmission beam and a second transmission beam sequentially, to a subject from the transducers. The receiver receives received signals that the transducers of the probe receive reflected waves of the first transmission beam and the second transmission beam respectively from the subject and output, and the receiver respectively beamforms received signals in the long axis direction of the probe to generate a first received beam signal and a second received beam signal. The image former generates frame data using the first received beam signal and the second received beam signal. The synthesizer comprises at least one of a signal synthesizer and an image synthesizer, the signal synthesizer weighting and synthesizing the first received beam signal and the second received beam signal, and the image synthesizer weighting and synthesizing a first frame data generated by the image former from the first received beam signal and a second frame data generated by the image former from the second received beam signal.

According to the present invention, it is possible to increase the resolution of the received signals in the short axis direction of the probe, by utilizing that the positions where the beams of the first and second transmission beams are focused from the transmit apertures having different sizes in the short axis direction of the probe, are different in the depth direction.

### BRIEF DESCRIPTION OF THE DRAWINGS

FIG. 1 is a block diagram showing an overall configuration of an ultrasound imaging apparatus according to a first embodiment;

FIG. 2A-1 and FIG. 2B-1 each illustrates an arrangement of transducers and driven transducers (transmit aperture) viewed from the upper surface of a probe used in the first embodiment, and FIGS. 2A-2, 2B-2 and 2C each illustrates the arrangement of the transducers viewed from the side surface of the probe in the short axis direction, the driven transducers (transmit aperture), and the shape of the first transmission beam 10;

FIG. 3 is a graph showing an example of weights used for weighting by a synthesizer according to the first embodiment;

FIG. 4 is a flowchart showing the operation of each unit when imaging is performed by a line data synthesis mode of the ultrasound imaging apparatus according to the first embodiment;

FIG. 5 illustrates a sequence when imaging is performed by the line data synthesis mode of the ultrasound imaging apparatus according to the first embodiment;

FIG. 6 is a flowchart showing the operation of each unit when imaging is performed by a frame data synthesis mode of the ultrasound imaging apparatus according to the first embodiment;

FIG. 7 illustrates the sequence when imaging is performed by the frame data synthesis mode of the ultrasound imaging apparatus according to the first embodiment;

FIG. 8 illustrates weights used for weighting the frame data by the image synthesizer in the first embodiment;

FIG. 9 is a flowchart showing the operation of each unit during imaging by the ultrasound imaging apparatus according to a second embodiment;

FIG. 10 illustrates the sequence when imaging is performed by the ultrasound imaging apparatus according to the second embodiment;

FIG. 11 is a flowchart showing the operation of each unit during imaging by the ultrasound imaging apparatus according to a third embodiment;

FIG. 12 illustrates the sequence when imaging is performed by the ultrasound imaging apparatus according to the third embodiment;

FIG. 13 illustrates the sequence when imaging is performed by the ultrasound imaging apparatus according to a fourth embodiment;

FIG. 14 is a flowchart showing the operation of each unit during imaging by the ultrasound imaging apparatus according to a fifth embodiment;

FIG. 15 illustrates the sequence when imaging is performed by the ultrasound imaging apparatus according to the fifth embodiment;

FIG. 16 is a flowchart showing the operation of each unit during imaging by the ultrasound imaging apparatus according to a sixth embodiment;

FIG. 17 illustrates a sequence when imaging is performed by the ultrasound imaging apparatus according to the sixth embodiment;

FIG. 18 illustrates the sequence fixing a combination of an angle and a aperture size in the short-axis direction of the probe for transmission during imaging by the ultrasound imaging apparatus according to the sixth embodiment;

FIG. 19 illustrates the sequence where the transmission is angled in five directions during imaging by the ultrasound imaging apparatus according to the sixth embodiment;

FIG. 20 illustrates the sequence fixing a combination of the angles in the five directions for the transmission and the aperture size in the short-axis direction of the probe during imaging by the ultrasound imaging apparatus according to the sixth embodiment;

FIG. 21 is a flowchart showing the operation of each unit when synthesizing received beam signals (RF data) having phase information during imaging by the ultrasound imaging apparatus according to the sixth embodiment;

FIG. 22 illustrates the sequence when synthesizing the received beam signals (RF data) having phase information during imaging by the ultrasound imaging apparatus according to the sixth embodiment;

FIG. 23 is a flowchart showing the operation of each unit when switching between the first transmit aperture and the second transmit aperture every transmission during imaging by the ultrasound imaging apparatus according to the sixth embodiment; and FIG. 24 illustrates the sequence when switching between the first transmit aperture and the second transmit aperture every transmission during imaging by the ultrasound imaging apparatus according to the sixth embodiment.

### DETAILED DESCRIPTION OF THE PREFERRED EMBODIMENTS

There will now be described embodiments of the present invention in detail with reference to the accompanying drawings. In all the figures for describing the embodiments, members having the same functions are denoted by the same reference numerals, and they will not be described redundantly. Further, in the following embodiments, the same or similar parts will not be described repeatedly in principle, except in cases of necessity.

In the drawings for describing the embodiments, for the purpose of clarifying the configuration, even a plan view may be provided with hatching, and on the other hand, hatching may not be provided even in a cross-sectional view.

### Embodiment 1

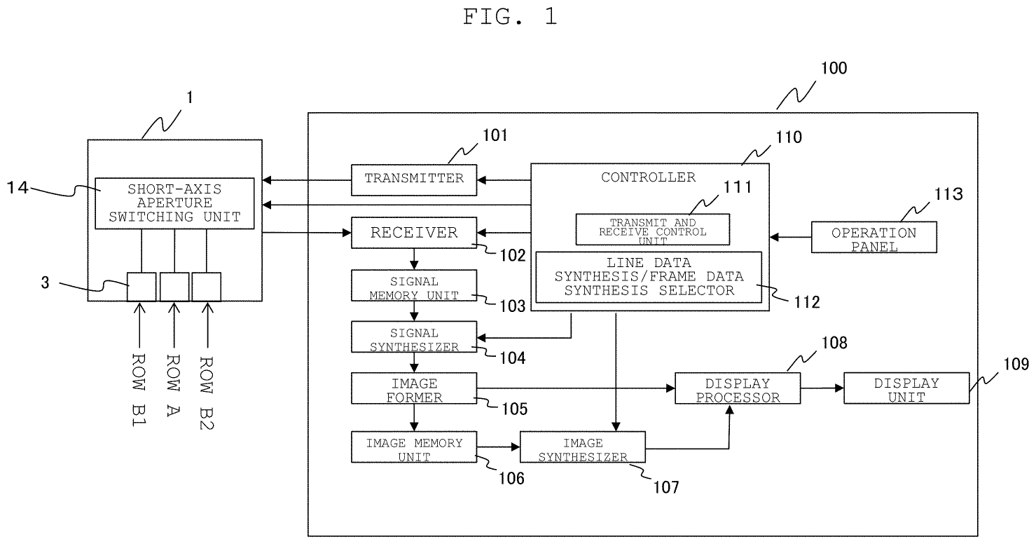

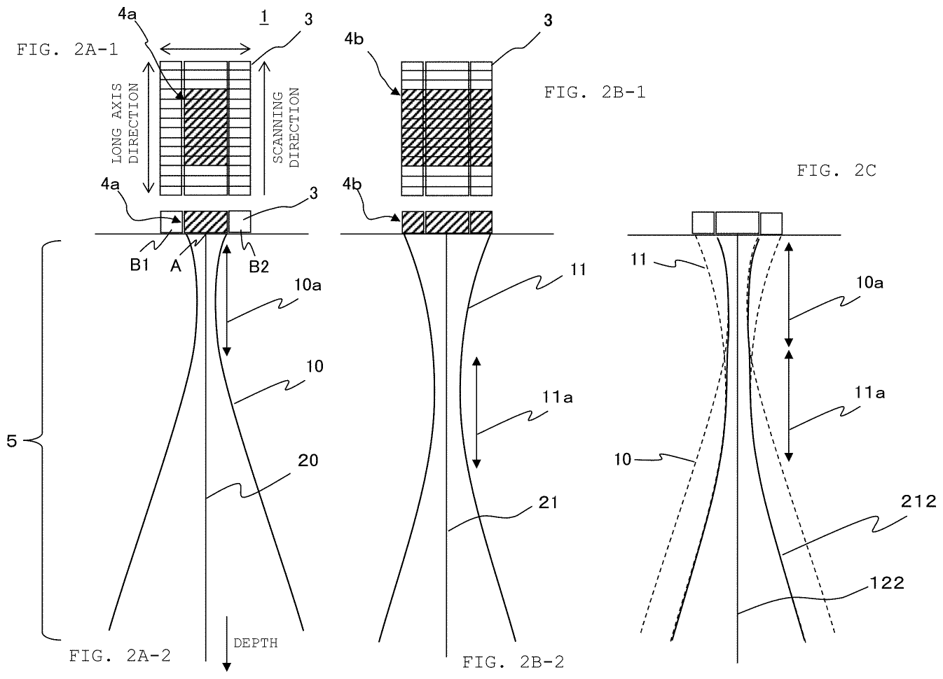

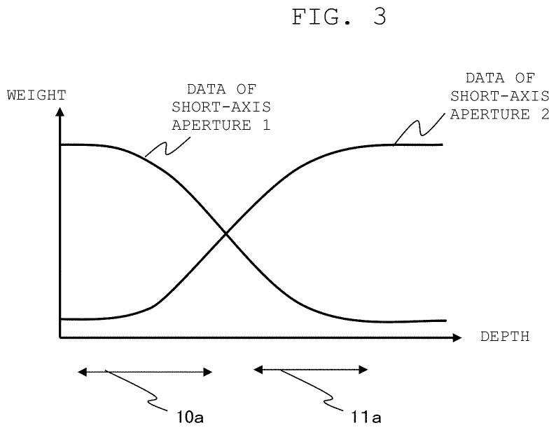

First, with reference to FIGS. 1 and 2, there will now be described a structure of an ultrasound imaging apparatus according to a first embodiment. FIG. 1 illustrates an overall configuration of the ultrasound imaging apparatus. FIG. 2A-1 and FIG. 2B-1 each illustrates an arrangement of transducers and driven transducers (transmit aperture) viewed from the upper surface of a probe, and FIGS. 2A-2, 2B-2 and 2C each illustrates the arrangement of the transducers viewed from the side surface of the probe, the driven transducers, and the shape of the first transmission beam.

First, there will be described a principle for obtaining a high-resolution image in the short axis direction of the probe, though the ultrasound imaging apparatus 100 has a simple configuration. As shown in FIGS. 1, 2A-1, and 2B-1, the ultrasound imaging apparatus 100 according to the present embodiment is connected to a probe 1 where the transducers 3 are arranged in the long axis direction and the short axis direction of the probe.

As shown in FIG. 1, the ultrasound imaging apparatus 100 comprises a controller 110 including a transmit and receive control unit 111 and a line data synthesis/frame data synthesis selector 112, a transmitter 101, a receiver 102, a signal memory unit 103 configured to store a received signal, a signal synthesizer 104, an image former 105, an image memory unit 106 configured to store image data, an image synthesizer 107, a display processor 108, a display unit 109, and an operation panel 113.

As shown in FIGS. 2A-1, 2A-2, 2B-1, and 2B-2, the transmitter 101 has a transmit aperture 4 provided in the probe 1, and delivers transmission signals to the transducers 3 in the transmit aperture 4, respectively. At this time, the transmitter 101 sequentially sets the first transmit aperture 4a having a predetermined size in the short-axis direction of the probe 1 and the second transmit aperture 4b having the size in the short-axis direction of the probe 1 larger than the first transmit aperture 4a, and transmits the first and second transmission beams 10 and 11 to the subject 5, from the transducers 3 respectively of the first and second transmit apertures 4a and 4b. Preferably, the center position of the first transmit aperture 4a in the short-axis direction of the probe 1 coincides with that of the second transmit aperture 4b.

As in FIG. 2A-2, the first transmission beam 10 transmitted from the first transmit aperture 4a having the smaller width in the short axis direction of the probe 1, has a beam width being narrowed at a predetermined depth position in the short axis direction of the probe 1. Thus in the first depth region 10a where the beam width is narrowed, the beam width of the short axis direction of the probe 1 is narrow.

On the other hand, as in FIG. 2B-2, the second transmission beam 11 transmitted from the second transmit aperture 4b having the larger width in the short axis direction of the probe 1, than the first transmit aperture 4a, has the beam width narrowed down in a predetermined second depth region deeper than the first transmission beam 10 in the short axis direction of the probe 1. Therefore, the second depth region 11a having a narrow beam width in the short axis direction of the probe 1 appears at a position deeper than the first depth region 10a having the narrow beam width of the first transmission beam 10.

The transducers of the probe 1 receive from the subject 5, the reflected waves of the first and second transmission beams 10 and 11. The receiver 102 receives the signals from the transducers 3, and with respect to the long direction of the probe 1, the receiver performs beamforming by delaying the received signals on the transducer basis and adding (delay-and-sum) those signals, so as to obtain the first and second received beam signals (beamformed signals) 20 and 21.

In the first and second depth regions 10a and 11a respectively irradiated with the first and second transmission beams 10 and 11 having been narrowed in the short-axis direction of the probe 1, signal resolution of each of the first and second received beam signals 20 and 21 is increased in the short axis direction of the probe 1.

The image former 105 generates image frame data using the first received beam signal 20 and the second received beam signal 21.

The synthesizer is provided with at least one of the signal synthesizer 104 and the image synthesizer 107. The signal synthesizer 104 weights and synthesizes the first received beam signal 20 and the second received beam signal 21 including the phase information of signals. The image synthesizer 107 weights and synthesizes the first image frame data generated by the image former 105 from the first received beam signals 20 and the second image frame data generated by the image former 105 from the second received beam signals 21.

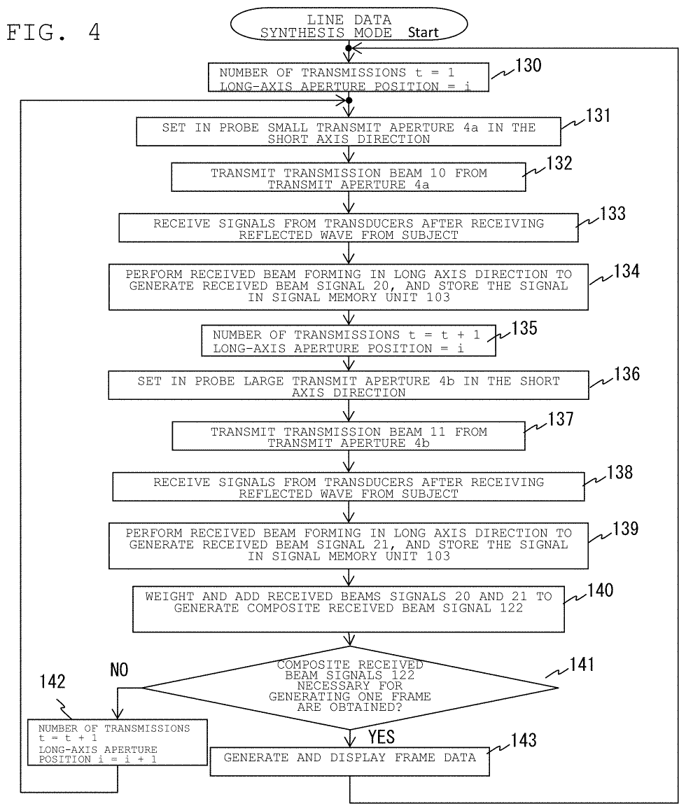

FIG. 3 illustrates an example of weighting in this situation. In the first depth region 10a where the depth in the subject 5 is shallow, the weight of the first received beam signal 20 or the first frame data is set to be larger than the weight of the second received beam signal 21 or the second frame data. Further, in the second depth region 11a where the depth in the subject 5 is large or in a deeper region, the weight of the second received beam signal 21 or the second frame data is set to be larger than the weight of the first received beam signal 20 or the first frame data.

However, depending on the relationship between the first and second transmit apertures 4a and 4b, and the short-axis focal point according to the lens, there may be a condition where the beam width in the short-axis direction of the first transmission beam again becomes narrower than the second transmission beam, at a depth larger than the second region 11a. Therefore, the weighting method is not limited to the example shown in FIG. 3, and it may be appropriately set according to the design. That is, the signal synthesizer 104 assigns the weight such that in the first depth region 10a where the depth in the subject is shallow, one of the first received beam signal 20 and the second received beam signal 21 is weighted larger than the other, whereas in at least a portion of the second depth region 11a deeper than the first depth region 10a, the other of the first received beam signal 20 and the second received beam signal 21 is weighted larger than the one. Similarly, the image synthesizer 107 assigns the weight in such a manner that, in the first depth region 10a where the depth in the subject is shallow, one of the first frame data and the second frame data is weighted larger than the other, and in at least a part of the second depth region 11a deeper than the first depth region 10a, the other of the first frame data and the second frame data is weighted larger than the one.

This synthesis process allows obtainment of a composite received beam signal 122 or composite frame data with a high signal resolution in the first and second depth regions 10a and 11a, as in the case of applying the combined beam 212 (see FIG. 2C) obtained by combining the first and second transmission beams 10 and 11. Accordingly, it is possible to obtain the composite received beam signal 122 or composite frame data, having higher resolution in the shorter axis direction of the probe 1, and uniform in the depth direction, on which the first and second received beam signals 20 and 21 are reflected, the signals being obtained by narrowing the first and second transmission beams 10 and 11 in the first and second depth regions 10a and 11a in the short axis direction of the probe 1.

In the example shown in FIG. 1, as the probe 1, there is employed the probe in which three transducers 3 are arranged in the short axis direction, or the probe in which three or more transducers 3 are arranged and divided into three regions (rows) in the short axis direction of the probe. Regarding the three rows of the transducers (or regions) in the short axis direction, the middle row is referred to as row A, and the rows on both sides thereof are referred to as rows B1 and B2. Though not illustrated, an acoustic lens is fixed to the surface of the probe 1 for outputting ultrasonic waves, and this acoustic lens allows the ultrasonic waves to converge in the short axis direction. A plurality of transducers in the short axis direction of the probe 1 may be arranged so that the surfaces for outputting the ultrasonic waves are curved instead of the acoustic lens, thereby allowing the ultrasonic waves to converge in the short axis direction, as in the case of the acoustic lens. The probe 1 may not be provided with the acoustic lens or the structure of curved arrangement of the transducers. Variations of the width of the transmit aperture in the short axis direction of the probe 1 can change the depth at which the width of the transmission beam is narrowed in the short axis direction of the probe 1.

Further, each of the transducers in the three rows of the probe 1 may be connected to a short-axis aperture switching unit 14. Upon transmission, the ultrasound imaging apparatus 100 switches the short-axis aperture switching unit 14, enabling selective input of transmission signals (electrical pulses) delivered from the ultrasound imaging apparatus 100, into the transducers 3 in at least one of the three rows. Further at the time of reception, the ultrasound imaging apparatus 100 switches the short-axis aperture switching unit 14. This allows selective inputting of only the received signals from the transducers 3 in one row, out of the received signals (electrical signals) of the ultrasonic waves received from the subject and outputted from the transducers 3 in the three rows, and it is further possible to add (short-circuit) the received signals of the transducers 3 in two or more rows to be inputted into the ultrasound imaging apparatus 100.

The probe 1 may not be provided with the short-axis aperture switching unit 14. In that case, at the time of transmission, the transmitter 101 selectively inputs transmission signals to the transducers in one or more three rows, thereby setting the first and second transmit apertures 4a and 4b. Upon reception, the receiver 102 selectively receives the signals received by the transducers in one or more of three rows, and adds the signals after the reception, thereby setting the receive aperture.

### <Operation of Each Unit to Perform Imaging>

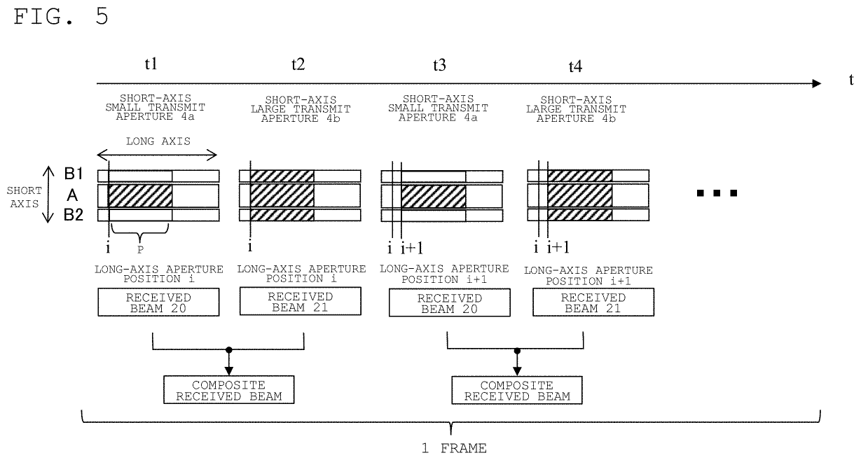

With reference to the flowcharts in FIGS. 4 and 6, and FIGS. 5 and 7 showing the transmit aperture for each transmission, there will now be described the operation of each unit when the ultrasound imaging apparatus 100 of the first embodiment takes an image of the subject 5.

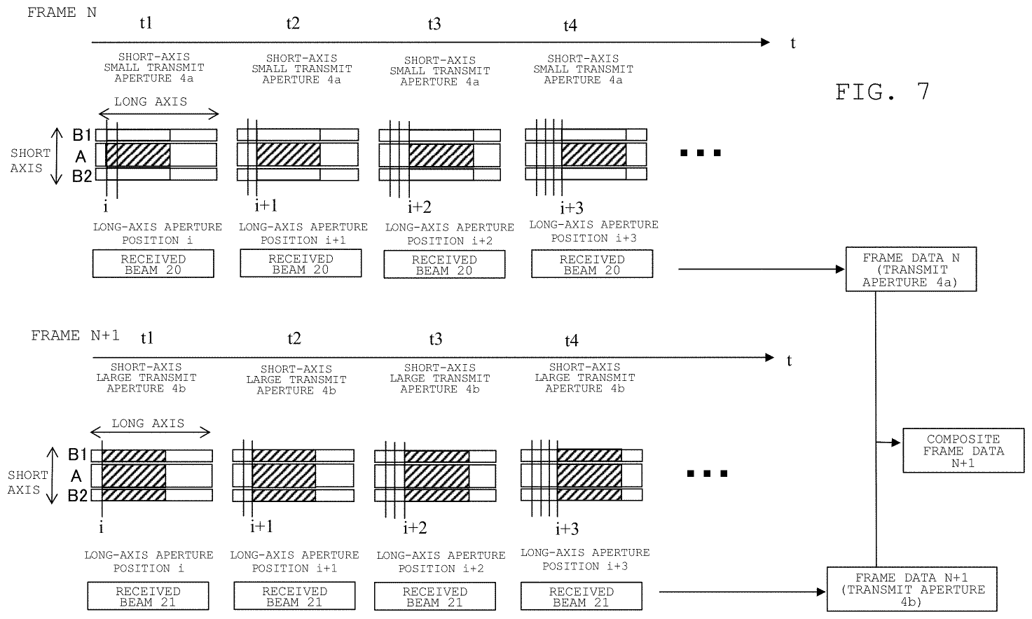

When selection of a line data synthesis mode from an operator is accepted via an operation panel 113, the line data synthesis/frame data synthesis selector 112 controls the operation of each unit according to the flowchart of FIG. 4, whereas it controls the operation of each unit according to the flowchart of FIG. 6 when selection of a frame data synthesis mode is accepted.

In the present embodiment, the transmitter 101, the receiver 102, the controller 110, and the signal synthesizer 104 can be configured by hardware. For example, a custom IC such as ASIC (Application Specific Integrated Circuit) or a programmable IC such as FPGA (Field-Programmable Gate Array) may be used for a circuit design to implement the functions of each unit. Functions of the transmitter 101, the receiver 102, the controller 110, and the signal synthesizer 104, may also be implemented partially and entirely in software. In this case, computer or a similar unit provided with a processor such as a CPU (Central Processing Unit) and a GPU (Graphics Processing Unit), and a memory, configures the transmitter 101, the receiver 102, the transmit and receive control unit 111, and the signal synthesizer 104, and the CPU reads and executes the programs stored in the memory, thereby implementing the configuration above.

### <Line Data Synthesis Mode>

There will now be described an example of operation of each unit, when the line data synthesis/frame data synthesis selector 112 accepts the selection of the line data synthesis mode.

### <Steps 130 and 131>

In the first transmission (t=1), the transmit and receive control unit 111 sets the first transmit aperture 4a of a small width in the short axis direction of the probe 1, at an aperture position i in the long axis direction of the probe 1. For example, the transmit and receive control unit 111 switches the short-axis aperture switching unit 14 of the probe 1, thereby selecting the transducers 3 located in the row A at the center in the short axis direction of the probe 1, as well as selecting a predetermined number of (e.g., P) transducers 3 from the aperture position i in the long axis direction, which are set as the first transmit aperture 4a. Alternatively, the transmit and receive control unit 111 sends an instruction to the transmitter 101 to set as the first transmit aperture 4a, the predetermined number of (e.g., P) transducers 3 from the position i in the long-axis direction of the probe 1 in the row A in the short-axis direction of the probe 1.

### <Step 132>

The transmitter 101 outputs transmission signals to the transducers 3 in the first transmit aperture 4a. Then, from the first transmit aperture 4a, the first transmission beam 10 is transmitted to the subject 5.

The depth region 10a where the beam width of the first transmission beam 10 in the short axis direction of the probe 1 is the narrowest appears at a shallow position.

In the long axis direction of the probe 1, the transmitter 101 provides a delay amount to each of the transmission signals outputted to the transducers 3 so as to focus the signals at a predetermined position. Therefore, the position where the beam width becomes the narrowest in the long axis direction of the probe 1 corresponds to thus provided focal position.

### <Step 133>

In response to the first transmission beam 10, ultrasonic waves reaching the probe 1, out of the ultrasonic waves such as reflected or scattered from the subject 5, are received by the transducers 3.

The receiver 102, here by way of example, receives signals from the transducers 3 within the first transmit aperture 4a. That is, the receiver 102 receives the signals from the transducers 3 in the central row A in the short axis direction of the probe 1, and the number of the transducers corresponds to a predetermined number (e.g., P pieces) from the aperture position i in the long axis direction of the probe 1. The transducers 3 from which the receiver 102 receives the signals are not limited to those within the transmit aperture. Any receive aperture different from the transmit aperture may be provided to receive the signals from the transducers 3 in the receive aperture, or it may also be possible to receive the signals from all the transducers 3 of the probe 1.

### <Step 134>

The receiver 102 performs receive-beamforming by delaying the received signals at a predetermined delay amount and adding the signals in the long axis direction of the probe 1, and generates a first received beam signal (also referred to as RF signal) 20 for a predetermined reception scanning line. The receiver 102 stores thus generated first received beam signal 20 in the signal memory unit 103.

The reception scanning line may be one line provided at the central position (position i+P/2) of the first transmit aperture 4a in the long axis direction. Alternatively, it is also possible to set a plurality of reception scanning lines around this one scanning line, and the first received beam signal may be generated for each.

### <Steps 135 and 136>

In the second transmission (t=2), the transmit and receive control unit 111 provides the second transmit aperture 4b having a large width in the short axis direction of the probe 1 at the position i in the same long axis direction as in step 131. For example, the transmit and receive control unit 111 switches the short-axis aperture switching unit 14 of the probe 1, thereby selecting the transducers 3 located in the rows A, B1 and B2 in the short-axis direction of the probe 1, together with selecting a predetermined number (e.g., P) of transducers 3 from the aperture position i in the long axis direction of the probe 1, so as to set the second transmit aperture 4b. The second transmit aperture 4b may be provided, alternatively, when the transmit and receive control unit 111 gives instructions to the transmitter 101 to set as the second transmit aperture 4b, the predetermined number (e.g., P) of the transducers 3 from the aperture position i in the long axis direction, and located in the rows A, B1 and B2 in the short axis direction.

### <Step 137>

The transmitter 101 outputs transmission signals to the transducers 3 within the second transmit aperture 4b. Then, the second transmission beam 11 is transmitted through the second transmit aperture 4b to the subject 5. The depth region 11a where the beam width of the second transmission beam 11 in the short axis direction of the probe 1 is the narrowest is deeper than the depth region 10a where the beam width of the first transmission beam 10 in step 132 is the narrowest.

### <Step 138>

In response to the first transmission beam 10, ultrasonic waves reaching the probe 1, out of the ultrasonic waves from the subject 5 such as reflected or scattered therefrom, are received by the transducers 3.

The receiver 102 receives signals from the transducers 3 in the second transmit aperture 4b, by way of example. That is, the receiver 102 receives the signals from the transducers 3 in the rows A, B1 and B2 in the short axis direction of the probe 1, and the number of the transducers corresponds to a predetermined number (e.g., P) from the aperture position i in the long axis direction of the probe 1. For the short axis direction, the short-axis aperture switching unit 14 short-circuits the transducers 3 in the rows A, B1 and B2, thereby outputting the summed received signals from the three transducers. Alternatively, the receiver 102 which has received the received signals from the transducers 3 in the rows A, B1 and B2, sums the received signals to be used for reception beamforming. Similar to step 133, the transducers 3 from which the receiver 102 receives the signals are not limited to those within the transmit aperture 4b. Any receive aperture different from the transmit aperture may be provided to receive the signals from the transducers 3 in the receive aperture, or it may also be possible to receive the signals from all the transducers 3 of the probe 1.

### <Step 139>

The receiver 102 performs receive-beamforming by delaying the received signals at a predetermined delay amount and adding (delay-and-sum) the signals in the long axis direction of the probe 1, and generates a second received beam signal (also referred to as RF signal) 21 for a predetermined reception scanning line. The receiver 102 stores thus generated second received beam signal 21 in the signal memory unit 103.

### <Step 140>

The signal synthesizer 104 reads the first received beam signal 20 and the second received beam signal 21 from the signal memory unit 103, adds those signals with assigning the weight as shown in FIG. 3, and generates a composite received beam signal 122. Thus, for the short axis direction of the probe 1, it is possible to obtain the composite received beam signal 122 with high resolution in the wide depth regions 10a and 11a.

### <Steps 141 and 142>

The transmit and receive control unit 111 repeats the above-described steps 131 to 140 until the number of composite received beam signals 122 necessary for creating one frame is obtained while shifting the positions of the first and second transmit apertures 4a and 4b in the long axis direction of the probe 1.

### <Step 143>

In step 141, when the composite received beam signals 122 are obtained, the number of which is required for creating one frame, the image former 105 generates the frame data from the composite received beam signals 122, and outputs the frame data to the display processing unit 108. The display processing unit 108 shows the frame data on the display unit 109.

According to the line data synthesis mode as described so far, the first received beam signal 20 obtained by transmitting the first transmission beam 10 from the first transmit aperture 4a having the small width in the short axis direction of the probe 1, and the second received beam signal 21 obtained by transmitting the second transmission beam 11 from the second transmit aperture 4b having the large width, are synthesized, and weighted in the depth direction. Accordingly, it is possible to display the frame data high in resolution in the short axis direction of the probe 1 and uniform in the depth direction.

In the aforementioned line data synthesis mode shown by the flowchart in FIG. 4, there has been described a configuration where the signal synthesizer 104 weights and synthesizes the first and second received beam signals 20 and 21 in the state of RF signals (signals having phase components). The present embodiment is, however, not limited to this configuration. As far as it is configured such that transmission is performed with varying the dimension size in the short axis direction of the probe 1 on each of scanning lines (reception scanning lines) and then obtained data of the reception scanning lines are synthesized, any of RF data and brightness data is available as the reception scanning line data to be synthesized. In other words, the received beam signals 20 and 21 may be converted to brightness data (absolute value data having no phase component) and thereafter synthesized. Specifically, for example, it is also possible to configure such that the image former 105 converts the received beam (line) signals 20 and 21 obtained from the transmission beams delivered through the first transmit aperture 4a and the second transmit aperture 4b that are set to the aperture position i in the long axis direction, into brightness (image) data for each of the reception scanning lines, and stores the brightness data in the image memory unit 106. Then the image synthesizer 107 weights and synthesizes the brightness data of the same line (reception scanning line) to generate the synthetic brightness data. Repeating the same process at the position (i+1), the synthetic brightness data for each line is stored in the image memory unit 106. When the synthetic brightness data of each line is accumulated as image data for one frame, this image data may be outputted to the display processing unit 108.

### <Frame Data Synthesis Mode>

Next, with reference to FIGS. 6 to 8, there will be described the operations of each unit in the case where the line data synthesis/frame data synthesis selector 112 accepts from an operator, a selection of the frame data synthesis mode. In the frame data synthesis mode, after the frame data is generated by transmitting through the first transmit aperture 4a having the small width in the short axis direction of the probe 1, another frame data is generated by transmitting through the second transmit aperture 4b having the large width, and those frame data items are weighted and synthesized.

### <Step 230>

According to steps 231 to 238 as described below, frame data imaging of frame N (N=1) is performed.

### <Steps 231 to 238>

Similar to steps 130 to 134 in the flowchart of the received beam synthesis mode as shown in FIG. 4, in steps 231 to 237, the transmit and receive control unit 111 sets in the probe 1 or the transmitter 101, the first transmit aperture 4a having the small aperture in the short axis direction of the probe 1 (step 231), and the transmitter 101 transmits the first transmission beam 10 (steps 231, 232). Then, the receiver 102 receives the reflected wave from the subject 5 and beamforms the reflected wave in the long axis direction of the probe 1, so as to generate the first received beam signal 20 (steps 234, 235). Unlike the line data synthesis mode of FIG. 4, however, the frame data synthesis mode of FIG. 6 repeats the transmission through the first transmit aperture 4a having the small width in the short axis direction in succession while shifting the aperture position in the long axis direction of the probe 1 as shown in FIG. 7 (step 237), and then the first received beam signals 20 of the number necessary for generating one frame is acquired (step 236).

The image former 105 uses thus obtained first received beam signals 20 to generate the frame data (for example, brightness data (image)) of the frame N (N=1), and stores thus generated frame data in the image memory unit 106 (Step 238).

### <Step 239>

The image synthesizer 107 assigns weights on the frame data of frame N and frame N−1 stored in the image memory unit 106 in the depth direction, and then synthesizes the data. In the case of initial Frame 1, N=1, the frame data of Frame N−1 is not stored in the image memory 106, and therefore the process proceeds directly to step 240.

### <Steps 240 and 241>

In order to perform imaging of the next frame N+1 (Frame 2), the transmit and receive control unit 111 switches the aperture size of the transmit aperture 4 in the short axis direction of the probe 1, between the small width and the large width, and then the process returns to step 232 (step 231). In the case of Frame 2, since the small width in the short axis direction of the probe 1 is set in Frame 1, it is switched to the second transmit aperture 4b having the large width in the short axis direction (see FIG. 7).

### <Steps 232 to 238>

The aforementioned steps 232 to 238 are repeated with the setting of the second transmit aperture 4b. That is, the process of transmitting the second transmission beam 11, receiving the reflected wave from the subject 5, and beamforming in the long axis direction of the probe 1 to generate the second received beam signal 21, is continuously repeated with shifting the aperture position in the long axis direction of the probe 1, thereby obtaining necessary number of second received beam signals 21 for generating one frame (steps 232 to 237). The image former 105 generates the frame data (image) of Frame 2 using thus obtained receive beam signals 21, and stores the frame data in the image memory unit 106 (Step 238).

### <Step 239>

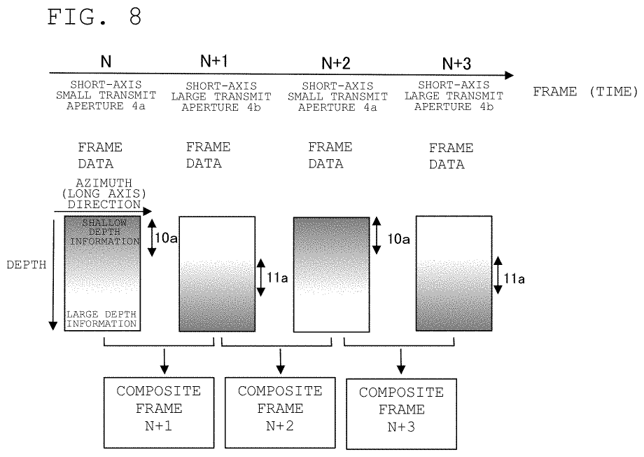

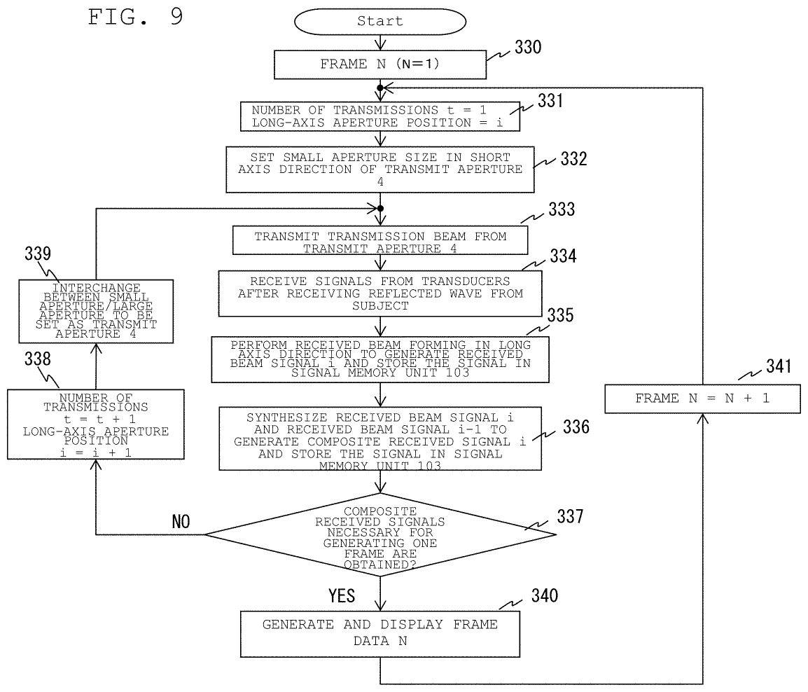

The image synthesizer 107 assigns weights in the depth direction and synthesizes the frame data of Frame 2 with the frame data of Frame 1 stored in the image memory unit 106. As shown in FIG. 8, the weights are uniform in the long axis direction of the probe 1, and are distributed in the depth direction as shown in FIG. 3. That is, in the region 10a where the depth is shallow, the weight of the frame data N obtained by setting the first transmit aperture 4a of the small aperture in the short axis direction of the probe 1 is made larger than the weight on the frame data N+1 obtained by setting the second transmit aperture 4b of the large aperture in the short axis direction of the probe 1. In the region 11a with a large depth or at a deeper point in the subject 5, the weight of the frame data N+1 obtained by setting the second transmit aperture 4b is set to be larger than the weight of the frame data N obtained by setting the first transmit aperture 4a.

The image synthesizer 107 outputs the composite frame data (frame data N+frame data N+1) to the display processing unit 108. The display processing unit 108 displays the frame data after the synthesis on the display unit 109.

Thus, according to the frame data synthesis mode, the frame data obtained by transmitting the first transmission beam 10 through the first transmit aperture 4a having the small width in the short axis direction of the probe 1 and the frame data obtained by transmitting the second transmission beam 11 through the second transmit aperture 4b having the large width are weighted in the depth direction and synthesized, whereby the frame data having high resolution in the short axis direction of the probe 1 and uniform in the depth direction can be displayed.

There has been described the configuration that in the frame data synthesis mode of the flowchart shown in FIG. 6 as described above, the image synthesizer 107 weights and synthesizes the frame data having been converted into brightness data (image data), but the present embodiment is not limited to this configuration. The frame data to be synthesized may be any data, RF data or brightness data, as far as the configuration is as the following; first, the width size in the short axis direction of the probe 1 is set to a certain size (small width or large width) to acquire data for one frame (data amount required for one image), and then, the aperture size in the short axis direction of the probe 1 is set to another size (large width or small width) to obtain one frame of data (data amount required for one image), followed by the process for synthesizing the data corresponding to thus obtained two frames. For example, the first transmission beam 10 is transmitted through the first transmit aperture 4a having the small width in the short axis direction of the probe 1 while sequentially shifting the transmit aperture 4a in the long axis direction of the probe 1, and thus obtained received beam signals 20 are stored in the signal memory unit 103, in the form of RF data corresponding to one frame. Next, the second transmission beam 11 is transmitted through the second transmit aperture 4b having the large width in the short axis direction of the probe 1 while sequentially shifting the transmit aperture 4b in the long axis direction of the probe 1, and thus obtained received beam signals 21 are stored in the signal memory unit 103, in the form of RF data corresponding to one frame. It is configured such that the signal synthesizer 104 weights and synthesizes the received beam signals 20 and 21 respectively for one frame, and the image former 105 converts the combined frame data into brightness data, to be outputted to the display processing unit 108.

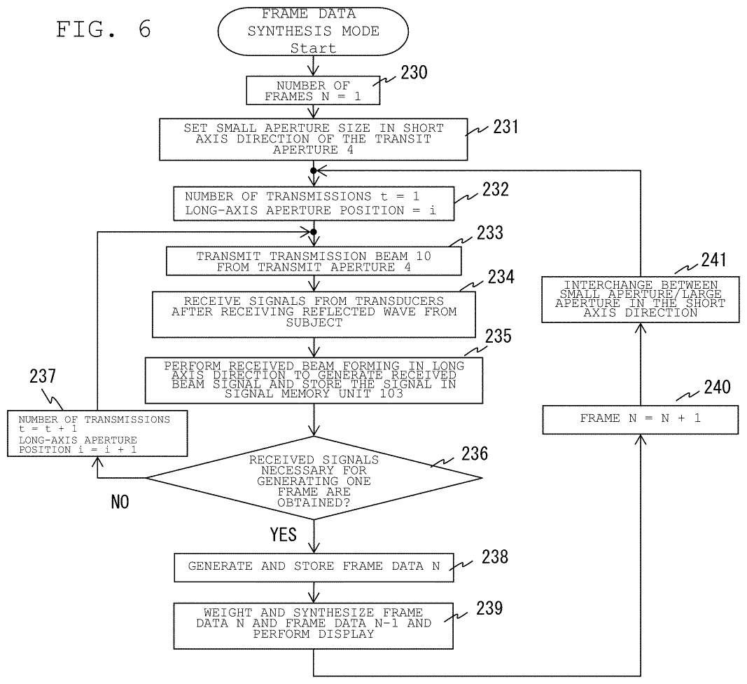

The frame data synthesis mode shown in FIGS. 6 and 7 has an advantage that reduction of the frame rate itself can be prevented by using one frame data and its previous frame data, in comparison with the line data synthesis mode shown in FIGS. 4 and 5.

The ultrasound imaging apparatus of the first embodiment is not necessarily provided with both the signal synthesizer 104 and the image synthesizer 107 at the same time. Either one of them may be sufficient.

In the first embodiment, the ultrasound imaging apparatus 100 and the probe 1 are separate devices, but the entire of or a part of the transmitter 101 and the receiver 102 of the ultrasound imaging apparatus may be configured to be placed within the probe 1. Further, the short-axis aperture switching unit 14 may be provided as a separate device outside the housing of the probe 1. Alternatively, the short-axis aperture switching unit 14 may be provided within the ultrasound imaging apparatus 100.

In the first embodiment, it is configured such that the receiver 102 receives the signals by the transducers 3 within the transmit aperture 4, but this is just an example. In the present invention, there is no essential difference if the signals are received from different transducers 3, not in the transmit aperture 4 and used for the receive beamforming.

Further, the number of divisions in the short axis direction in the probe 1 is not limited to three.

In the above-described line data synthesis mode, there has been described the configuration for combining the received beam signals after delaying and adding (delay-and-sum) (see step 140 in FIG. 4). It is also possible to synthesize the received data (channel data) obtained from the transducers 3. Specifically, it is sufficient that the received data (channel data) acquired from the transducers 3 in step 133, and the received data (channel data) acquired in step 138 are synthesized in step 140 for each corresponding transducer (channel), and the received data after the synthesis is subjected to beamforming in the same manner as in step 139.

### Embodiment 2

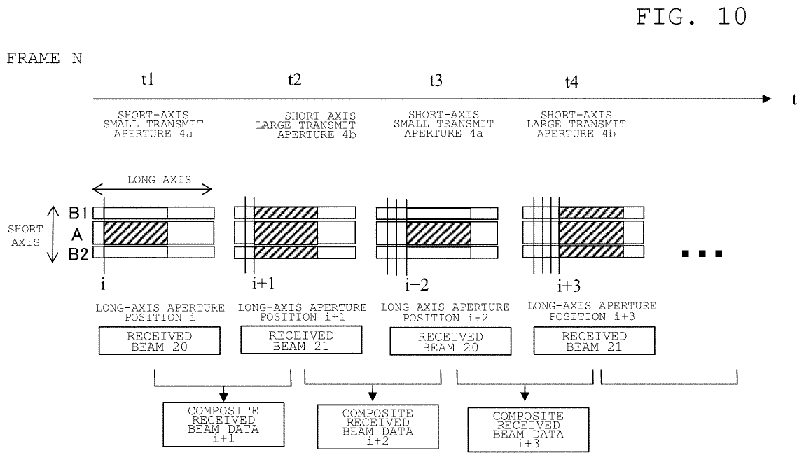

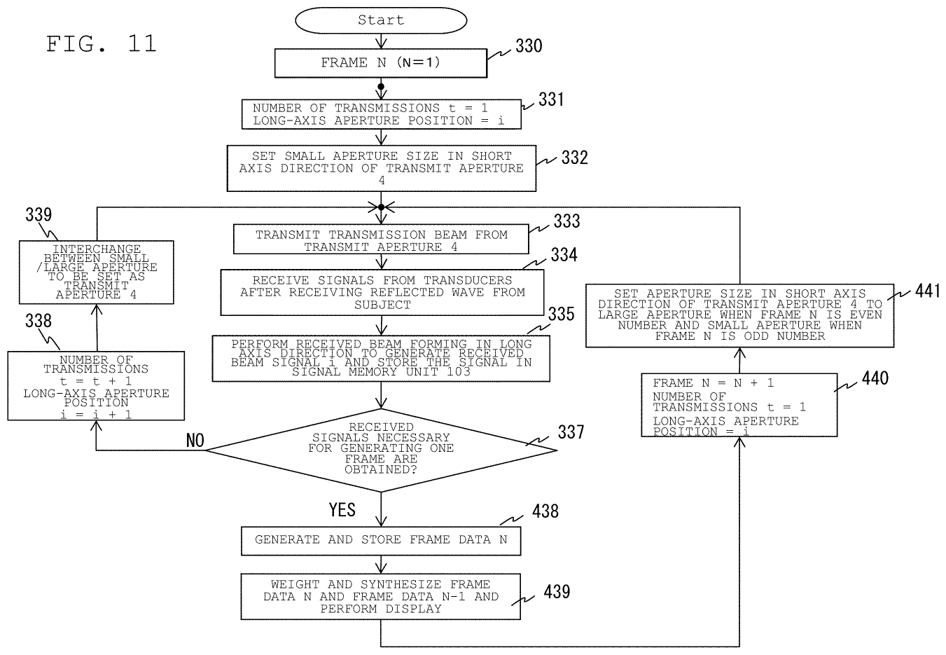

With reference to FIGS. 9 and 10, there will be described an operation at the time of imaging by the ultrasound imaging apparatus according to a second embodiment. Since the configuration of the ultrasound imaging apparatus of the second embodiment is the same as in the first embodiment, redundant descriptions will not be provided.

In the second embodiment, in order to obtain the frame data of one frame N, each time moving the transmit aperture 4 in the azimuth direction (long axis direction) of the probe 1, the aperture size in the short-axis direction of the probe 1 is switched between the small width and the large width. Thus, the first and second received beam signals adjacent to each other in the azimuth direction of the probe 1 are obtained by setting either the first transmit aperture 4a or the second transmit aperture 4b, different in aperture size in the short axis direction of the probe 1. In the present embodiment, the first and second received beam signals 20 and 21 being adjacent are weighted and combined in the depth direction, thereby forming a composite beam signal in one azimuth direction.

By repeating this operation in transmitting and receiving in the azimuth direction, it is possible to obtain a frame data having high resolution in the short axis direction and excellent uniformity in the depth direction without reducing the frame rate.

In addition, since the received beam signals 20 and 21 having different transmit dimension sizes in the short axis direction of the probe 1 are equally included in one frame, this achieves high trackability to the probe operation and motion of the living body, thereby presenting synchronized information between a shallow portion and a deep portion.

With reference to FIGS. 9 and 10, there will be specifically described the operation of the ultrasound imaging apparatus according to the second embodiment.

### <Step 330>

The frame data of frame N (N=1) is generated in the following steps 331 to 340.

### <Steps 331 to 335>

Similar to steps 130 to 134 of the line data synthesis mode as shown in the flowchart of FIG. 4, in steps 331 to 335, at the first transmission t=1, the transmit and receive control unit 111 sets the first transmit aperture 4a having the small width in the short axis direction to the aperture position i in the long axis direction of the probe 1 (steps 331 and 332). The transmitter 101 transmits the first transmission beam 10 through the first transmit aperture 4a (step 333), the receiver 102 receives from the transducers 3, signals obtained by receiving the reflected wave from the subject 5, beamforms the received signals in the long axis direction of the probe 1 to generate a first received beam signal (i) 20, and stores the signal in the signal memory unit 103 (step 334, 335).

### <Step 336>

The signal synthesizer assigns weights in the depth direction and then synthesizes the received beam signal (i) and the adjacent received beam signal (i−1) stored in the signal memory unit 103 to obtain the composite received beam signal (i), and stores the composite received beam signal in the signal memory unit 103. In the case of the initial i=1 receive beam signal (i), the receive beam signal (i−1) is not stored yet, and therefore the process proceeds directly to step 337.

### <Step 337>

The transmit and receive control unit 111 determines whether or not the composite receive beam signals (i) are obtained, the number of which is required for creating one frame, and if not obtained, the process proceeds to step 338.

### <Steps 338 and 339>

At the second transmission t=2, the second transmit aperture 4b having the large width in the short axis direction is provided at the aperture position i+1 in the long-axis direction of the probe 1, the aperture size of the second transmit aperture 4b being switched from the first transmission, and the process returns to step 333.

### <Steps 333 to 335>

In steps 333 to 335, the transmitter 101 transmits the second transmission beam 11 through thus provided second transmit aperture 4b having the large width in the short-axis direction of the probe 1, and the receiver 102 receives the signals from the transducers 3, beamforms the received signals in the long axis direction of the probe 1 to generate the received beam signal (i+1) 21, and stores the received beam signal in the signal memory unit 103.

### <Step 336>

The signal synthesizer assigns weights as shown in FIG. 3 in the depth direction and synthesizes the received beam signal (i+1) and the adjacent received beam signal (i) stored in the signal memory unit 103 to obtain a composite receive beam signal (i+1), and stores it in the signal memory unit 103.

### <Step 337>

The above steps 331 to 336 are repeated while shifting the aperture position in the long axis direction of the probe 1, until the composite receive beam signals (i) are obtained, the number of which is required for creating one frame.

### <Step 340>

When the composite received beam signals are obtained, the number of which is necessary for creating one frame, the image former 105 generates the frame data (image) of the frame N (N=1) and outputs the frame data (image) to the display processing unit 108. The display processing unit 108 displays the frame data on the display unit 109.

### <Step 341>

The frame number is incremented, and the process returns to step 331 to repeat the above process.

Thus, in the second embodiment, as to each received beam signal of multiple received beam signals forming one frame data, at different positions in the long axis direction of the probe 1, two adjacent received beam signals are weighted in the depth direction and synthesized to form a composite beam signal in one azimuth direction. Therefore, it is possible to obtain the frame data having high resolution in the short axis direction of the probe 1 and excellent uniformity in the depth direction, without reducing the frame rate. Also, the received beam signals with different transmit dimension sizes in the short axis direction of the probe 1 are equally contained in one frame, achieving high trackability to the probe operation and motion of the living body, thereby presenting synchronized information between the shallow portion and the deep portion.

Also in the second embodiment, similar to the first embodiment, the received beam signals to be synthesized to generate the composite received beam signal is not limited to RF signal, and it is also possible to synthesize the signals after converting the received beam signal to brightness data.

### Embodiment 3

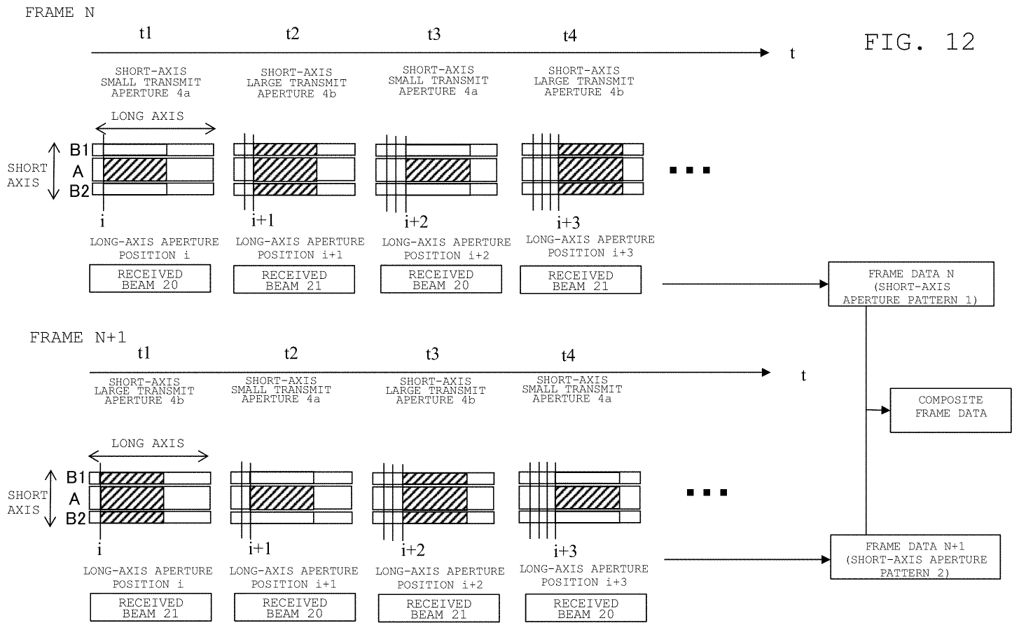

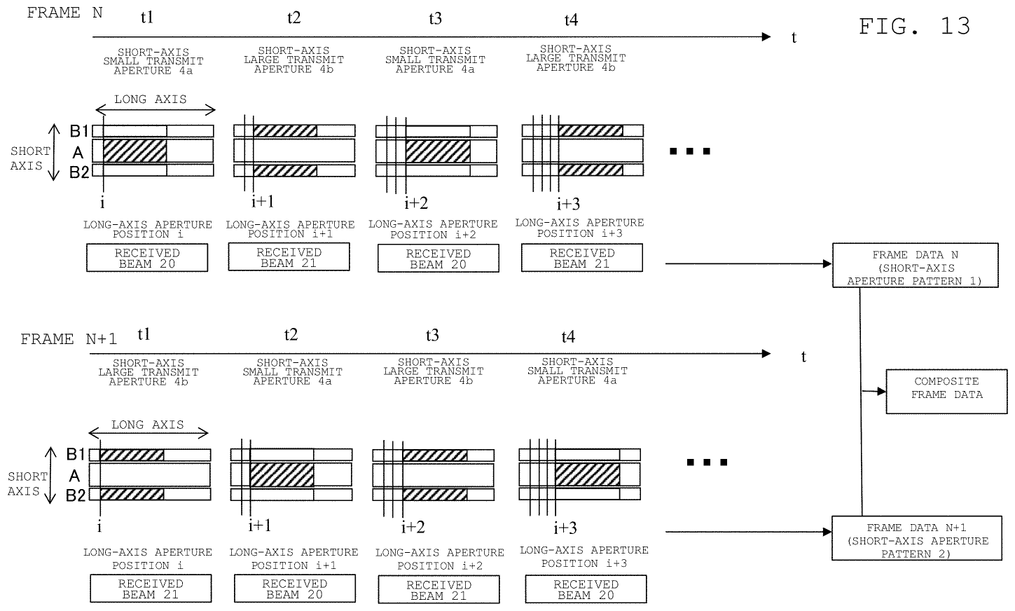

With reference to FIGS. 11 and 12, there will be described an operation at the time of imaging by the ultrasound imaging apparatus according to a third embodiment. As is apparent from FIGS. 11 and 12, the operation for the imaging in the ultrasound imaging apparatus according to the third embodiment includes many points common to FIGS. 9 and 10 according to the second embodiment. Thus, the same steps are denoted by the same step numbers, and only different points will be described. Further, the configuration of the ultrasound imaging apparatus according to the third embodiment is the same as the configuration of the first embodiment.

As shown in FIGS. 11 and 12, similarly to FIGS. 9 and 10 of the second embodiment, in the imaging operation of the ultrasound imaging apparatus according to the third embodiment, at the first transmission of the first frame N (N=1), the small width is set as the aperture size of the transmit aperture 4 in the short axis direction of the probe 1 (steps 330 to 333), and each time the transmit aperture 4 is moved in the azimuthal direction (the long axis direction), the aperture size of the transmit aperture 4 in the short axis direction of the probe 1 is switched between the small width and the large width (steps 338 and 339). This switching is repeated alternately to obtain the first and second received beam signals 20 and 21 necessary to generate one frame data (steps 334, 335, and 337).

Here, in the third embodiment, unlike the second embodiment, thus obtained first and second received beam signals 20 and 21 are used to generate the frame data N, and this frame data N is stored in the image memory unit 106 (step 438).

In the next frame N+1, the aperture size in the short-axis direction of the probe 1 is switched each time shifting the transmit aperture 4 in the azimuth direction (long axis direction) as in the previous frame N. In this case, at the same position in the long-axis direction as the previous frame N, the aperture size is made different from the aperture size in the short-axis direction provided in the previous frame N. That is, at the position where the small width was set as the aperture size in the short axis direction in the previous frame N, the transmit aperture having the large width in the short axis direction is set in the frame N+1. Further, in the previous frame N, at the position where the large width was set as the aperture size in the short axis direction, the transmit aperture having the small width in the short axis direction is set in the frame N+1.

In order to achieve the aforementioned operation, at the first transmission (t=1) for the frame N+1, the transmit aperture having the large width is set in the short axis direction, different from the first transmission of the previous frame (steps 440 and 441). Therefore, after incrementing the frame number N to N=N+1 in step 440, in step 441, when the frame number N after increment (=N+1) is an even number, the second transmit aperture 4b having the large width in the short axis direction is set, and when it is an odd number, the first transmit aperture 4a having the small width in the short axis direction is set. Thereafter, steps 333 to 339 are repeated to obtain the first and second received beam signals 20 and 21 to generate the frame data N(=N+1), and the generated frame data is stored in the image memory unit 106 (step 438).

The image synthesizer 107 assigns weights in the depth direction and synthesizes the frame N(=N+1) generated in step 438 with the frame data of the previous frame (N−1) stored in the image memory unit 106 (step 439). The weights are assigned in the depth direction for each scan line (received beam) constituting each frame data. Specifically, as in FIG. 3, in the region where the depth is shallow, the weight of the first received beam signal 20 obtained by setting the first transmit aperture 4a is made larger, whereas in the deeper region, the weight of the second received beam signal 21 obtained by setting the second transmit aperture 4b is made larger. The weighting shown here is just an example, and it may be appropriately configured according to the shape of the short-axis beam that is variable depending on design values.

The image synthesizer 107 outputs the composite frame data to the display processing unit 108. The display processing unit 108 displays the composite frame data on the display unit 109 (step 439).

Thus, in the present embodiment, by synthesizing the two frame data items, it is possible to obtain the same composite frame data as the data obtained by synthesizing the first and second received beam signals 20 and 21.

Similar to the frame data synthesis mode of FIGS. 6 and 7 according to the first embodiment, in the imaging method of the present embodiment, it is possible to display the frame data with high resolution in the short axis direction of the probe 1 and uniform in the depth direction, without reducing the updating rate of the frame rate.

Further, according to the imaging method of the present embodiment, one frame data before the synthesis includes information of the first and second received beam signals 20 and 21 equally, obtained by setting the transmit apertures with different aperture sizes in the short axis direction of the probe 1, and thus there is an advantage of high trackability for the motion of the subject 5.

Furthermore, according to the present embodiment, the two frame data items are synthesized, using the transmission and reception data items acquired through the transmit apertures set at the same position in the long axis direction of the probe 1, and thus the occurrence of artifacts can be reduced with high image quality, as compared with the image obtained according to the second embodiment.

In the third embodiment, similar to the first embodiment, the frame data to be synthesized is not limited to the image data converted into brightness data, and the data may be synthesized in the form of frame data where the received beam signals (RF data) are arranged.

### Embodiment 4

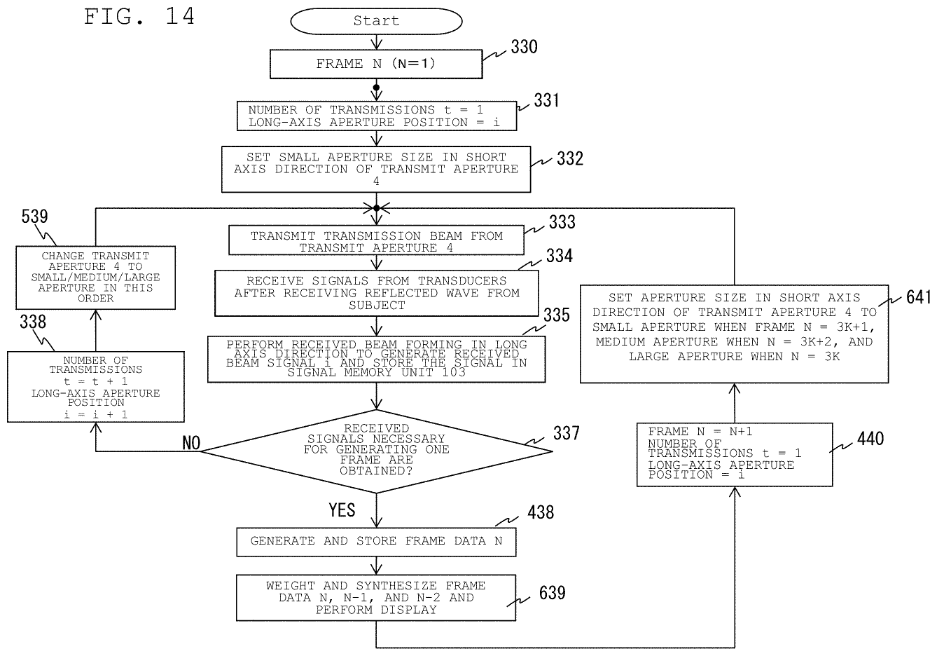

With reference to FIG. 13, there will be described an operation at the time of imaging by the ultrasound imaging apparatus according to a fourth embodiment.

In the first to third embodiments, the transducers 3 in the row A located at the center in the short axis direction of the probe 1 are selected as the first transmit aperture 4a having the small width in the short axis direction. As the second transmit aperture 4b having the large width in the short axis direction of the probe 1, the transducers 3 in the row A in the short axis direction and the transducers 3 in the row B1 and B2 adjacent to both sides of the row A are selected. It is to be noted, however, the present invention is not limited to these first and second transmit apertures 4a and 4b. Any aperture shape may be available as the first and second transmit apertures 4a and 4b, as far as the position where the beam width of the first and second transmission beams 10 and 11 transmitted from the first and second transmit apertures 4a and 4b are narrowed in the short axis direction of the probe 1 are different in the depth direction.

For example, as shown in FIG. 13, as the first transmit aperture 4a having the small width in the short axis direction of the probe 1, the transducers 3 in the row A are selected in the short axis direction, and as the second transmit aperture 4b having the large width in the short axis direction, only the rows B1 and B2 may be selected, without including the row A. In the case of the second transmit aperture 4b where only the rows B1 and B2 are selected in the short axis direction of the probe 1, the transducers in the row A at the center are not selected. Therefore, the signal intensity near the probe 1 (transducers 3) is reduced down, but the second transmission beam 11 is narrowed down at a distance. Thus, the second transmission beam 11 through the second transmit aperture 4b is narrowed down at a position deeper than the position where the first transmission beam 10 through the first transmit aperture 4a selecting the transducers 3 in the row A in the short axis direction is narrowed down. Accordingly, also in present embodiment, the same effect as in the first to third embodiments can be exerted.

The operation of each unit during the imaging as shown in FIG. 13 is the same as the flowchart of FIG. 11 of the ultrasound imaging apparatus according to the third embodiment, so redundant descriptions will not be given. It is also to be noted that the configuration of the ultrasound imaging apparatus of the fourth embodiment is the same as that of the first embodiment.

Further, using the first and second transmit apertures 4a and 4b as shown in FIG. 13, it is of course possible to execute the imaging method according to the first and second embodiments.

### Embodiment 5

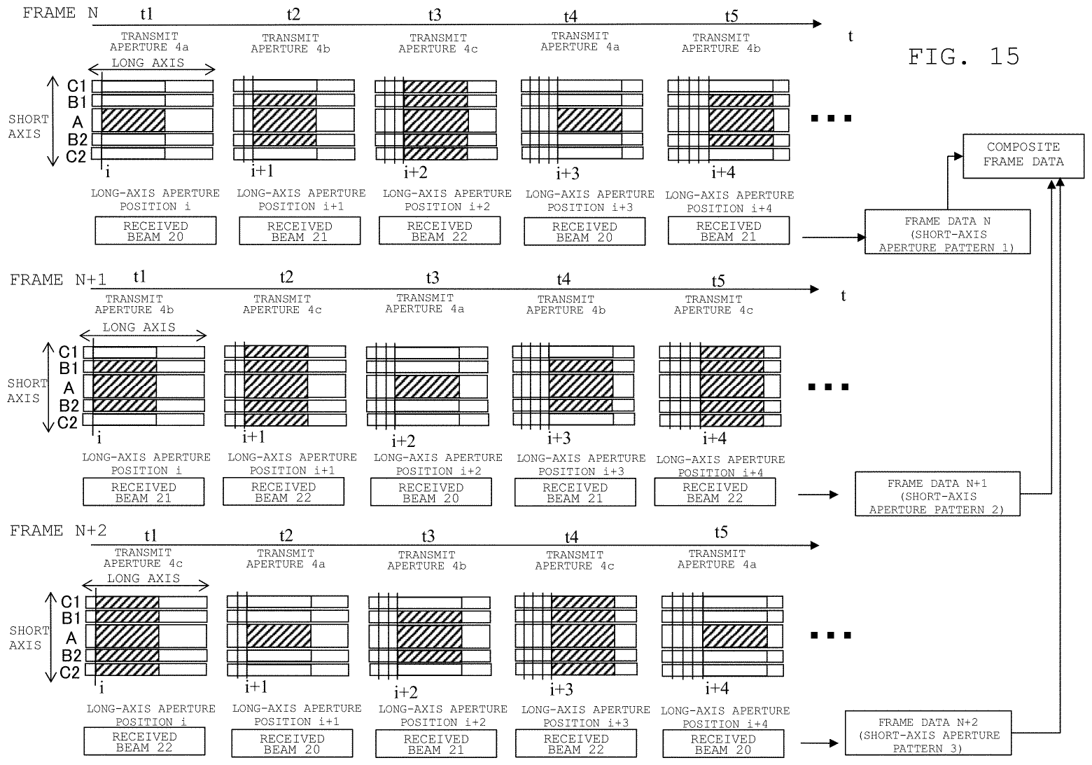

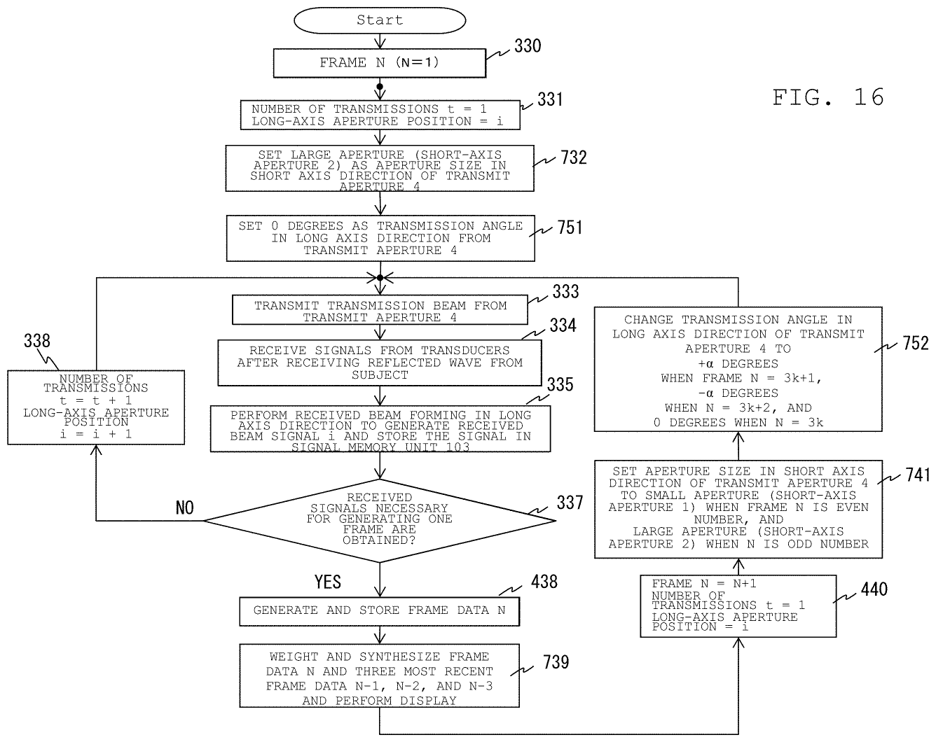

With reference to FIGS. 14 and 15, there will be described the ultrasound imaging apparatus according to a fifth embodiment.

In the first to fourth embodiments, for the sake of convenience, the number of transducers 3 in the short axis direction of the probe 1 (the number of divisions) is set to three, and the first transmitting aperture 4a having the small width and the second transmitting aperture 4b having the large width are provided. However, the number of the transducers 3 (the number of divisions) in the short axis direction of the probe 1 is not limited to three. FIG. 15 shows an example of an imaging operation in the case where the number of transducers 3 in the short axis direction (division number) of the probe 1 is five. The probe comprises the transducers in five rows; the row A, rows B1 and B2 adjacent to both sides of the row A, and the rows C1 and C2 adjacent to further both sides in the short axis direction.

As the transmit aperture in the short axis direction of the probe 1, there may be provided three types of transmit aperture, as an example; the first transmit aperture 4a selecting only the row A, the second transmit aperture 4b selecting the row A and the rows B1 and B2, and the transmit aperture 4c selecting all the rows (row A+rows B1 and B2+rows C1 and C2). Here, the first transmit aperture 4a is referred to as a small width, the second transmit aperture 4b is referred to as a medium width, and the transmit aperture 4c is referred to as a large width.

As shown in FIGS. 14 and 15, the operation at the time of imaging by the ultrasound imaging apparatus according to the present embodiment is the same as the operation at the time of imaging as shown in FIGS. 11 and 12 of the third embodiment. However, steps 539, 639 and 641 are different from the third embodiment.

In any of the frames, as in step 539, the transmit and receive control unit 111 sets the aperture size of the transmit aperture in the short-axis direction of the probe 1, in the order of the small width, the medium width, and the large width. Then, there are acquired received beam signals, the number of which is required for creating one frame. In this case, the transmit and receive control unit 111 sets the transmit aperture such that the received beam signals at the same position can be obtained respectively with setting the transmit apertures 4a, 4b, and 4c having different width in the short axis direction, in the frame N, the next frame N+1, and further in the next frame N+2. That is, the transmit and receive control unit 111 sets the transmit apertures in the order of the small width, the medium width, and the large width in the frame N, in the order of the medium width, the large width, and the small width in the next frame (N+1), and further in the next frame (N+2), in the order of the large width, the small width, and the medium width.

To achieve the operation above, in steps 332 and 641, the transmit and receive control unit 111 sets the aperture size of the transmit aperture at the transmission number t=1 as the top of each frame, in such a manner that the width is small in Frame 1, medium in Frame 2, and large in Frame 3. That is, the small width is set when the value of N of the frame N is represented by N=3k+1, the medium width is set when the value of N is represented by N=3k+2, and the large width is set when the value of N is represented by N=3k. Here, the value of k is an integer.

Further, at the time of each frame imaging, the transmit and receive control unit 111 performs switching of the aperture size of the transmit aperture in the short-axis direction in the order of the small width, the medium width, and the large width (step 539), every time shifting the transmit aperture in the long axis direction of the probe 1 (step 338).

Furthermore, the image former 105 generates the frame data N from the received beam signals being obtained (step 438), and the image synthesizer 107 weights and synthesizes the present frame data N, the previous frame data N−1, and the further previous frame data N−2. The image synthesizer 107 weights the received beam signals in the depth direction on a scanning line basis (received beam), the beam signals being obtained from each of the transmit apertures 4a, 4b, and 4c, in such a manner that the weight of the received beam signals obtained from one transmit aperture becomes larger in the region where the beam width of the transmission beam is narrowed in the short axis direction of the probe, relative to the received beam signals obtained from the other transmit apertures. Specifically, in the region where the depth is shallow, the weight of the first received beam signal 20 obtained by setting the first transmit aperture 4a is maximized, in the middle depth region, the second received beam signal 21 obtained by setting the second transmit aperture 4b is maximized, and in the region where the depth is large, the weight of the third received beam signal 22 obtained by setting the third transmit aperture 4c is maximized. The manner of weighting described here is one example, and the weight may be appropriately assigned according to the shape of the short-axis beam that may vary depending on the design value.

In the present embodiment, the image synthesizer 107 needs for the synthesis, the received beam signals 20, 21, and 22 obtained by setting the transmit apertures 4a, 4b, and 4c respectively having different aperture sizes in the short axis direction of the probe 1, and those signals may be obtained in no particular order. Therefore, the transmit and receive control unit 111 may change the setting order of the transmit apertures 4a, 4b, and 4c of each frame. For example, the transmit and receive control unit 111 may configure the settings in the frame N in the order of the first transmit aperture 4a (row A only), the third transmit aperture 4c (rows A+B1+B2+C1+C2), and the second transmit aperture 4b (rows A+B1+B2).

Further, in step 539, when the number of frames synthesized by the image synthesizer 107 is three, the effective frame rate may be reduced. In order to prevent the frame rate reduction, the transmit and receive control unit 111, for example, does not use the first transmit aperture 4a, and obtains frame data by alternately setting the second transmit aperture 4b (rows A+B1+B2) and the transmit aperture 4c (row A+B1+B2+C1+C2), so that the image synthesizer 107 can synthesize the two frame data items. Similarly, it may be configured such that the first transmit aperture 4a (row A) and the transmit aperture 4c (rows A+B1+B2+C1+C2) are set alternately, and the two frame data items are synthesized. There is an alternative configuration that even when the number of the transducers 3 in the short axis direction of the probe (the number of divisions) is five, the transmit and receive control unit 111 uses only two sets, out of the row A, the rows B1+B2, and the rows C1+C2.

As described so far, in the present embodiment, there is no need for an essentially different technique, particularly, depending on the number of transducers 3 (the number of divisions) in the short-axis direction of the probe. It is only required to increase the combinations of rows of the transducers 3 in the short-axis direction to be used, and the number of combinations may be appropriately changed depending on the application.

Further, the present embodiment is of course applicable to the first and second embodiments.

In the fifth embodiment, similar to the first embodiment, the frame data to be synthesized is not limited to the image data converted into brightness data, and the data may be synthesized in the form of frame data where the received beam signals (RF data) are arranged.

### Embodiment 6

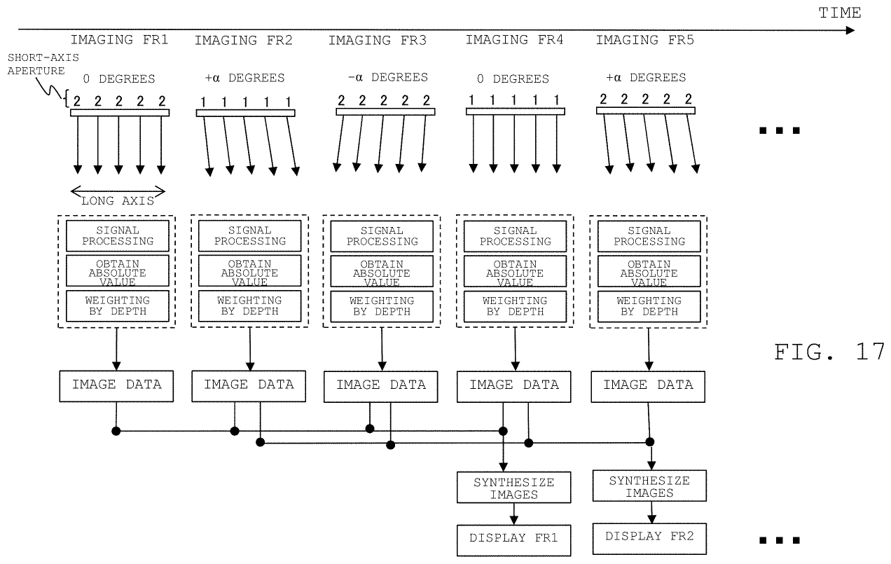

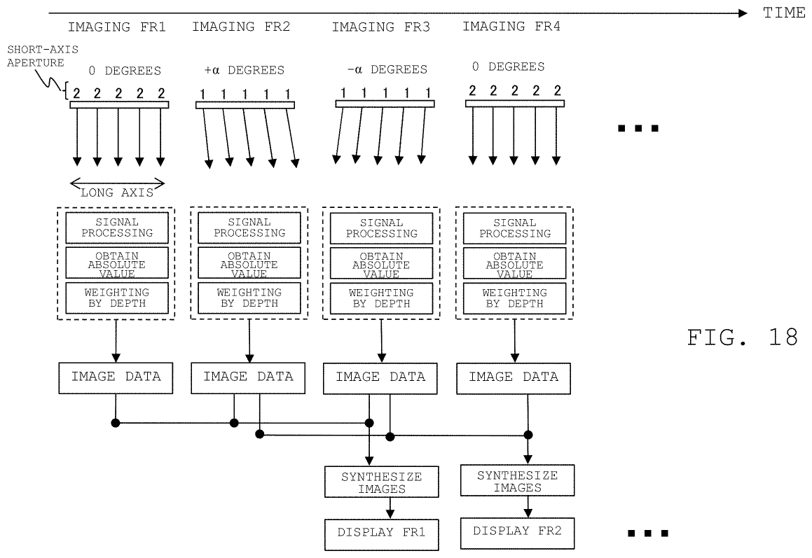

With reference to FIGS. 16 and 17, there will now be described the ultrasound imaging apparatus according to a sixth embodiment.

There is generally known an ultrasound imaging apparatus that varies the angle of emission of the transmission beam into a plurality of types, in a cross section including the long axis of the probe, and by synthesizing the received beam signals or frame data being obtained, thereby improving an image quality. This function is referred to as angular compound or spatial compound, for example.

In the present embodiment, there will now be described the ultrasound imaging apparatus using both the angular compound and the technique of the present invention for varying the transmit aperture in the short axis direction of the probe into multiple types of apertures. For the angular compound, it is necessary to emit transmission beams in the cross section including the long axis direction and the depth direction, respectively at multiple angles (three directions in the examples of FIGS. 16 and 17; 0 degrees, +α degrees, and −α degrees with respect to the depth direction), so as to acquire image data. Therefore, it takes imaging time as compared with the case of not using the angular compound. Therefore, if the transmission and reception with different short-axis aperture sizes are simply combined with the angular compound, i.e., the transmission of the transmission beam at a certain angle is performed through the transmit apertures of multiple types of short-axis aperture sizes, the time required for imaging will be further increased. In order to avoid this situation, in the present embodiment, the transmit and receive control unit 111 switches the aperture size in the short axis direction, simultaneously with switching the angle for transmission.

With reference to FIGS. 16 and 17, there will now be described the operation of each unit during imaging by the ultrasound imaging apparatus according to the present embodiment. In the flowchart of FIG. 16, the steps common to the flowchart of the fifth embodiment in FIG. 14 are assigned the same step numbers, and will not be described redundantly.

Here, there will be described the case where the number of transducers 3 (the number of divisions) in the short axis direction of the probe 1 is three. In the present embodiment, the first transmit aperture 4a having the small width selecting only the row A in the short axis direction, is referred to as the short axis width 1 and the second transmit aperture 4b having the large width selecting the rows A, B1, and B2 in the short axis direction are referred to as the short axis width 2.

As shown in FIGS. 16 and 17, in the initial transmission (t=1) of the first frame N (N=1), the transmit and receive control unit 111 sets the large width (short axis width 2) as the aperture size of the transmit aperture 4 in the short axis direction of the probe 1 (steps 330, 331, and 732), and further sets the transmission angle to 0 degrees in the long axis direction from the transmit aperture 3 (step 751). While the transmit and receive control unit 111 shifts the position of the transmit aperture in the long axis direction, the transmitter 101 repeats transmission of the transmission beam, the receiver 102 acquires the received beams necessary for generating one frame, and the image former 105 generates the frame data, at the angle of 0 degrees through the second transmit aperture 4b with the short-axis width 2 (large width) (steps 333 to 339 and 438).

Next, the transmit and receive control unit 111 increments the frame N to N=2 (step 440), switches the aperture size of the transmit aperture 4 in the short axis direction to the small width (short-axis width 1) (step 741), and the transmission angle to +α degrees (step 752). In order to make the transmission angle +α degrees, the transmitter 101 adjusts the delay time of the transmission signals to be outputted to the transducers 3 in the transmit aperture 4 in the long axis direction of the probe. Then, the process returning to step 333, while the transmit and receive control unit 111 shifts the position of the transmit aperture in the long axis direction, the transmitter 101 repeats the transmission of the transmission beam, the receiver 102 acquires the received beams necessary for generating one frame, and the image former 105 generates the frame data, at the angle of +α degrees through the first transmit aperture 4a with the short axis width 1 (small width) (steps 333 to 339, and 438).

Next, the transmit and receive control unit 111 increments the frame N to N=3 (step 440), switches the aperture size of the transmit aperture 4 in the short axis direction of the probe to the large width (short axis width 2) (step 741), and switches the transmission angle to −α degrees (step 752). Then, the process returning to step 333, while the transmit and receive control unit 111 shifts the position of the transmit width in the long axis direction of the probe, the transmitter 101 repeats the transmission of the transmission beams, the receiver 102 acquires the received beams necessary for generating one frame, and the image former 105 generates the frame data, at the angle of −α degrees through the second transmit aperture 4b with the short axis width 2 (large width) (steps 333 to 339, 438).

That is, in the present embodiment, in step 741, the aperture size in the short axis direction of the transmit aperture 4 is switched in accordance with the value of N of the frame N. Specifically, when N is an even number, it is switched to the first transmit aperture 4a having the small width (short axis width 1), and when N is an odd number, it is switched to the second transmit aperture 4b having the large width (short axis width 2).

Next, in step 752, the transmission angle of the transmission beam of the transmit aperture 4 in the long axis direction of the probe is switched according to the value of N of the frame N. Specifically, when N is represented by N=3k+1, it is switched to +α degrees with respect to the depth direction, when represented by N=3k+2, it is switched to −α degrees, and when N is represented by N=3k, it is switched to 0 degrees. Here, k is an integer.

The image synthesizer 107 reads the frame data N generated in step 438 and the three most recent frame data N−1, N−2, and N−3, from the image memory unit 106, and then weights and synthesizes these frame data items. Similarly to step 239 of the first embodiment, the weight is assumed as corresponding to the aperture size of the transmit aperture in the short axis direction of the probe set at the time of transmission (see FIG. 8). The display processing unit 108 displays thus composite frame data on the display unit 109 (step 739). The weighting may be performed on the frame data in the form of arranged RF data before generating the image data, or the weighting may be performed after the image data is generated.

As described so far, in the present embodiment, the image data obtained by each imaging frame is synthesized in this manner, and this allows simultaneous processing; the angular compound and synthesizing the frame data items respectively obtained through the transmit apertures of multiple types of aperture sizes in the short axis direction of the probe. Therefore, it is possible to perform two synthesizing operations without increasing the time required for imaging.

When the angular compound includes three directions and the type of the aperture size in the short-axis direction of the probe includes two stages, there may occur asymmetry in the frame data signals after synthesis, when the image synthesizer 107 combines three frame data items at different angles. This is because volume of the frame data obtained by setting the transmit aperture with either one of the two-stage aperture sizes may become larger than the frame data obtained by setting the transmit aperture with the other aperture size. In order to avoid this situation, in the example of FIG. 17, the image synthesizer 107 performs the synthesis process using four most recent frame data items.

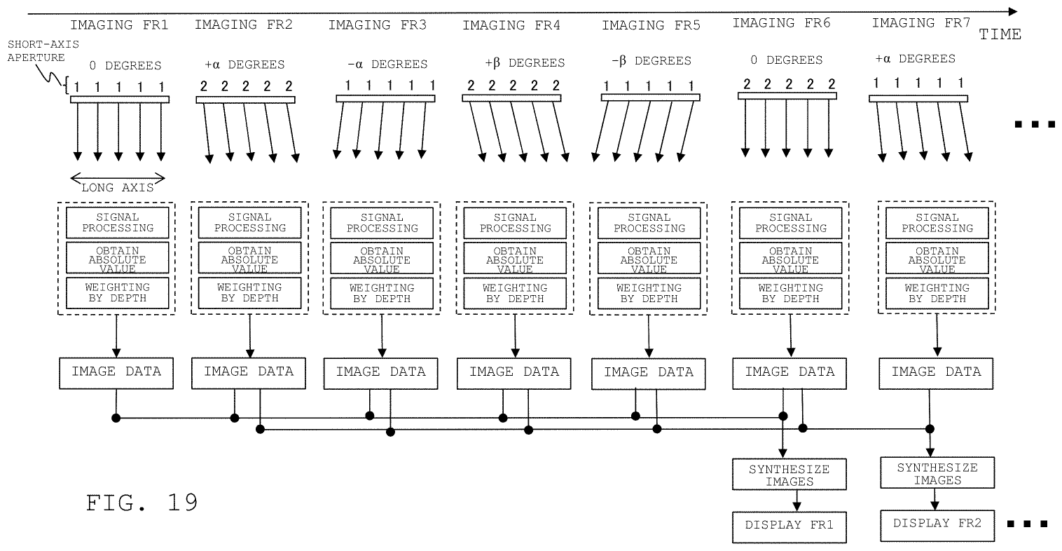

In FIGS. 16 and 17, the transmit and receive control unit 111 has the configuration that the transmission angle and the aperture size in the short-axis direction of the probe are switched every time the frame number is incremented, but this is not the only configuration. For example, as shown in FIG. 18, the transmit and receive control unit 111 may fix the combination of the transmission angle and the aperture size in the short axis direction. As an example, the transmit and receive control unit 111 sets the second transmit aperture 4b of the short axis width 2 (large width) when the transmission angle is 0 degrees, and sets the first transmit aperture 4a of the short axis width 1 (small width) when the transmission angle is ±α degrees. By fixing the combination of the transmission angle and the aperture size in the short axis direction in this way, it is possible to avoid the asymmetry of the aperture size in the short axis direction with respect to the angle. Changing the transmission angle in the order of 0 degrees, +α degrees, and −α degrees may cause that the short axis width 1 (small width) continues in the two frames of +α degrees and −α degrees. In the example of FIG. 18, however, the frames of three angles (0 degrees, +α degrees, and −α degrees) necessary for the angular compound are weighted and synthesized, without increasing the number of frames to be synthesized by the image synthesizer 107. Similar to step 239 of the first embodiment, the weight corresponds to the aperture size of the transmit aperture in the short axis direction as set at the time of transmission.

Also in the example of FIG. 18, the combination of the angle and the aperture size in the short axis direction may of course be changed.

The angle of the angular compound is not limited to three directions. As shown in FIG. 19, for example, the angle may be increased to five directions (0 degrees, +α degrees, −α degrees, +β degrees, −β degrees). In the example of FIG. 19, similar to FIG. 17, each time the frame number is incremented, the transmit and receive control unit 111 switches the transmission angle, and also switches the aperture size of the transmit aperture in the short axis direction at the same time. In FIG. 19, the image synthesizer 107 synthesizes six frames to obtain a composite image, so that the frames to be synthesized include the same number of frames between the frames with the short axis width 1 (small width) and the frames with the short axis width 2 (large width). For the other improvements such as the frame rate, however, the image synthesizer 107 may synthesize five frames.

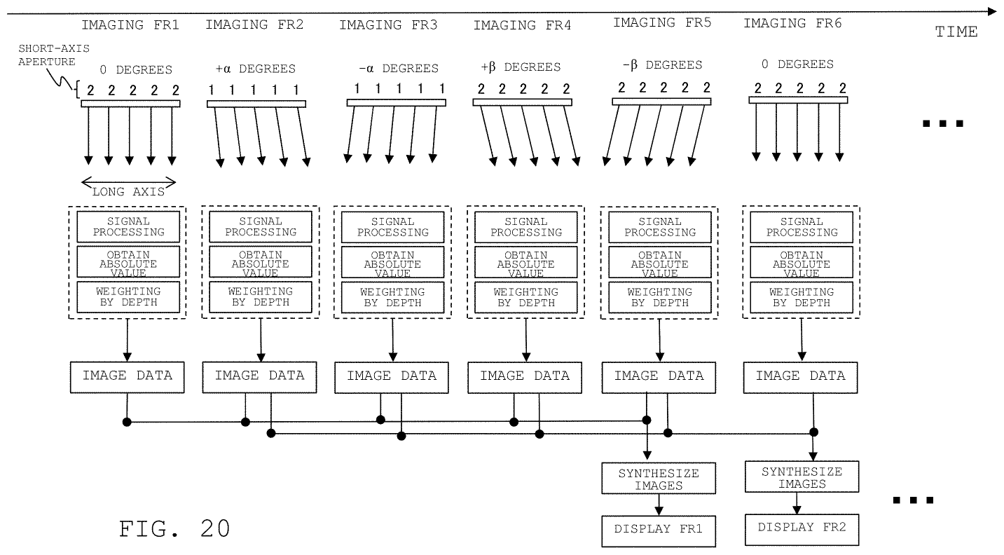

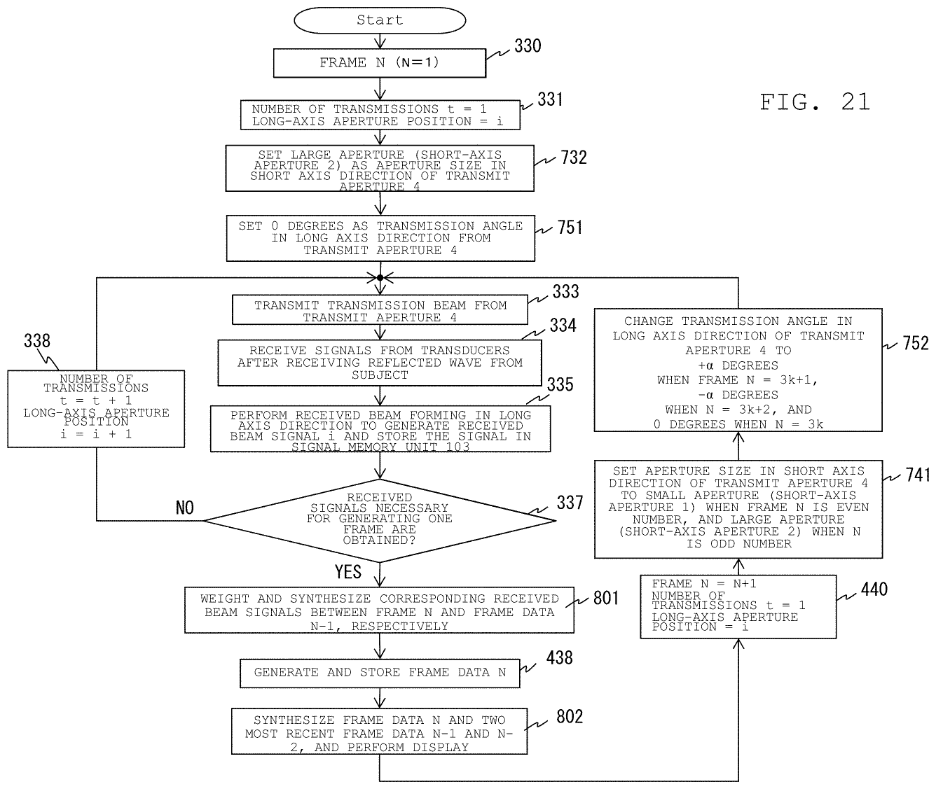

In the example shown in FIG. 20, the transmission angle includes five directions (0 degrees, +α degrees, −α degrees, +β degrees, and −β degrees), and similarly to FIG. 18, the combination of the transmission angle and the aperture size of the transmit aperture in the short axis direction is fixed. FIG. 20 shows an example, when the transmission angles are 0 degrees and ±β degrees, the transmit aperture is set to the short axis width 2 (large width), and when the angle is ±α degrees, the transmit aperture is set to the short axis width 1 (small width).

The combination of the transmission angle and the aperture size of the transmit aperture in the short-axis direction of the probe may be appropriately changed depending on the application and effect.

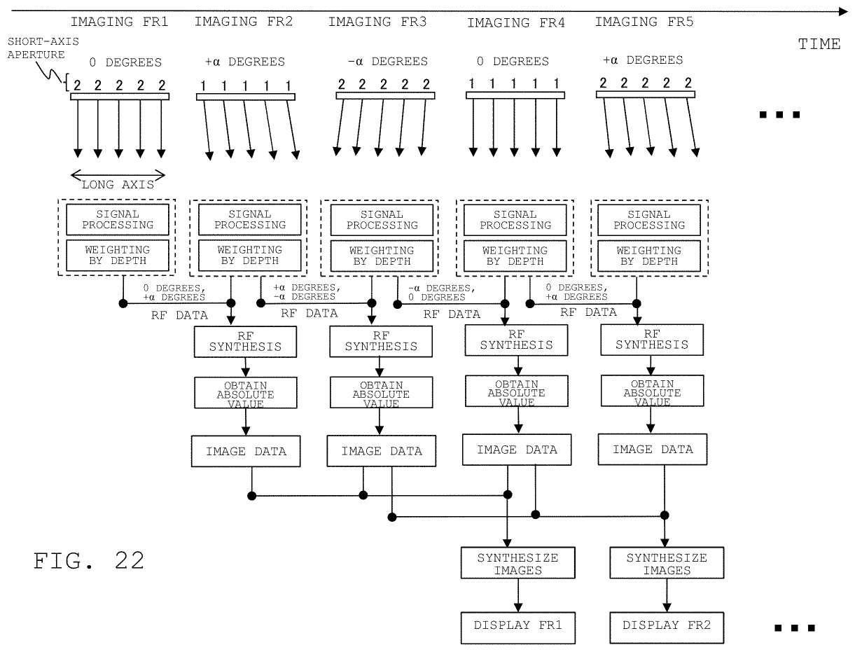

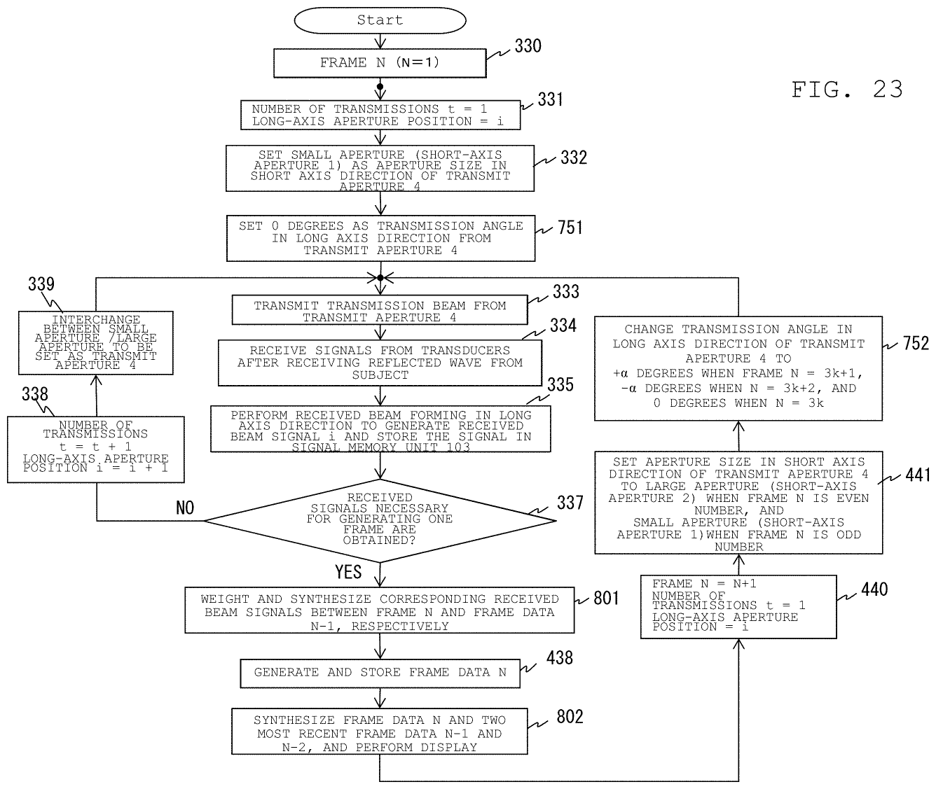

In FIGS. 16 to 20, the image synthesizer 107 has the configuration in which all the synthesis processing is performed using the image data (the absolute values of frame data). However, it is also possible to configure such that the received beam signals obtained by setting the transmit apertures having different size in the short axis direction of the probe are synthesized at the stage when the received beam signals have phase information (RF data). For example, as shown in FIGS. 21 and 22, similarly to FIGS. 16 and 17, the transmit and receive control unit 111 switches each of the transmission angle and the aperture size in the short axis direction each time the frame number is incremented, and the process of step 801 is performed between steps 337 and 438.

In step 801, the signal synthesizer 104 weights the received beam signals of the frame N and the corresponding received beam signals of the frame data N−1 respectively and synthesizes these signals. As shown in FIG. 3, the signal synthesizer 104 configures the settings of the weight, according to the aperture size of the transmit aperture in the short axis direction. Thus, it is possible to synthesize the received beam signals obtained by setting the transmit apertures having different transmit width in the short axis direction, at the stage of the received beam signals (RF data) with phase information.

The image former 105 performs processing such as arranging the absolute values of the received beam signals synthesized by the signal synthesizer 104, thereby generating image data (frame data), and stores the image data in the image memory unit 106 (step 438).

The image synthesizer 107 synthesizes the frame data N stored in the image memory unit 106, with the frame data items N−1 and N−2 of the previous two times, and displays the synthesized data (step 802). Accordingly, this enables the angular compound.

In the flowchart of FIG. 21, the same steps as in FIG. 16 are denoted by the same step numbers, and they will not be described redundantly.

According to the imaging operation of FIGS. 21 and 22, the received beam signals obtained by setting different aperture sizes in the short axis direction can be synthesized in the form of RF data, while the angular compound can be formed with the image data.

The combination of the transmission angle and the aperture size of the transmit aperture as shown in FIG. 22 is only one example, and it may be appropriately changed.

Further, in FIG. 22, there is shown the configuration that the transmission angle and the aperture size in the short-axis direction of the probe are switched each time the frame number is incremented, and similarly to the example of FIG. 18, the combination of the transmission angle and the aperture size of the transmit aperture may be fixed. The transmission angle in three directions may be increased to five directions or more. In addition, the aperture size in the short axis direction of the transmit aperture may have three stages, increased from two stages.

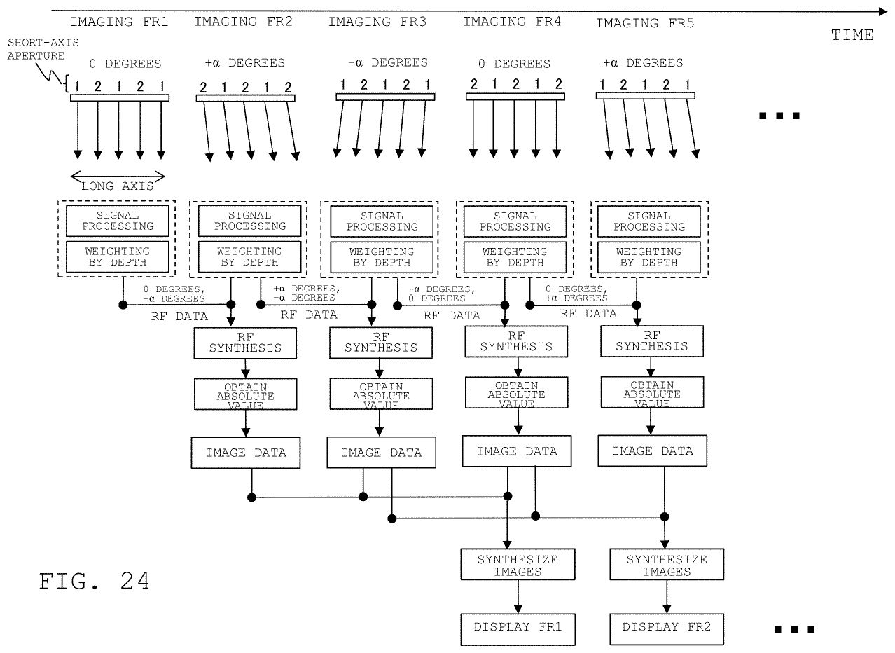

In the imaging operation shown in FIGS. 23 and 24, similarly to FIGS. 21 and 22, the signal synthesizer 104 synthesizes the received beam signals in the form of RF data, the received beam signals being obtained by the transmit and receive control unit 111 by setting different aperture sizes in the short axis direction. However, the transmit and receive control unit 111 has the configuration such that the aperture size in the short axis direction is switched every transmission and reception, similarly to FIG. 11, and this point is different from the configuration of FIGS. 21 and 22.

That is, in step 339 of FIG. 23, the transmit and receive control unit 111 switches, in the same manner as in FIG. 11, the aperture size in the short axis direction of the transmit aperture, between the small width (short axis width 1) and the large width (short axis width 2) for every transmission and reception, and in step 441, for each frame, the aperture size in the short axis direction of the transmit aperture of the first transmission, is switched between the small width (short axis width 1) and the large width (short axis width 2). With this configuration, the received beam signals obtained by setting the transmit aperture having the small width (short axis width 1) and the received beam signals obtained by setting the transmit aperture having the large width (short axis width 2) are included alternately in one frame, achieving improvement of the effective frame rate.

In the flowchart of FIG. 23, the same steps as in the flowchart of FIG. 21 are denoted by the same step numbers, and they will not be described redundantly.

Further, the combination of the transmission angle and the aperture size of the transmit aperture shown in FIG. 23 is just an example, and it may be changed appropriately. Alternatively, the combination of the transmission angle and the aperture size of the transmit aperture may be fixed. It is also possible to increase the number of directions of the transmission angle to five or more. In addition, the aperture size of the transmit aperture in the short axis direction may be increased to three or more stages instead of two stages.

Also in the imaging operation of FIGS. 23 and 24, the number of directions of the transmission angle may be more than three. In addition, any change of the short-axis aperture size, the number of stages, and the combination thereof will cause no essential difference.

As discussed above, the synthesis of the received beam signals obtained by setting different aperture sizes in the short axis direction of the transmit aperture may be performed with RF data including phase information, or may be performed with the absolute values of image data. There may be some differences, for example in texture, between the images obtained with the two types of data. Thus, the user is allowed to select with which data the synthesis is performed, RF data or image data, by manipulating the operation panel 113, and then the line data synthesis/frame data synthesis selector 112 performs control to switch the operations between the signal synthesizer 104 and the image synthesizer 107. Accordingly, the user can select an optimal synthesis method appropriately.

What is claimed is:

1. An ultrasound imaging apparatus comprising,
a transmitter, a receiver, an image former, and a synthesizer, wherein
the transmitter sequentially sets a first transmit aperture and a second transmit aperture in a probe where transducers are arranged in each of a long axis direction and a short axis direction of the probe, the first transmit aperture having a predetermined aperture size in the short axis direction of a probe and the second transmit aperture having the aperture size in the short axis direction of the probe larger than the first transmit aperture, the transmitter outputting transmission signals to the transducers in each of the first transmit aperture and the second transmit aperture, thereby transmitting a first transmission beam and a second transmission beam sequentially, to a subject from the transducers,
the receiver receives received signals that the transducers of the probe receive reflected waves of the first transmission beam and the second transmission beam respectively from the subject and output, and the receiver respectively beamforms received signals in the long axis direction of the probe to generate a first received beam signal and a second received beam signal,
the image former generates frame data using the first received beam signal and the second received beam signal, and
the synthesizer comprises at least one of a signal synthesizer and an image synthesizer, the signal synthesizer weighting and synthesizing the first received beam signal and the second received beam signal, and the image synthesizer weighting and synthesizing a first frame data generated by the image former from the first received beam signal and a second frame data generated by the image former from the second received beam signal.

2. The ultrasound imaging apparatus according to claim 1, wherein
the synthesizer weights one of the first received beam signal and the second received beam signal, larger than the other in a first depth region where the depth in the subject is shallow, whereas in at least a portion of a second depth region deeper than the first depth region, the other of the first received beam signal and the second received beam signal is weighted larger than the one.

3. The ultrasound imaging apparatus according to claim 1, wherein
the synthesizer weights one of the first frame data and the second frame data, larger than the other in a first depth region where the depth in the subject is shallow, whereas in at least a portion of a second depth region deeper than the first depth region, the other of the first frame data and the second frame data is weighted larger than the one.

4. The ultrasound imaging apparatus according to claim 1, wherein
the synthesizer weights the first received beam signal or the first frame data, larger than the second received beam signal or the second frame data in a first depth region where the depth in the subject is shallow, whereas in at least a portion of a second depth region deeper than the first depth region, the second received beam signal or the second frame data is weighted larger than the first received beam signal or the first frame data.

5. The ultrasound imaging apparatus according to claim 1, wherein
the transmitter sets the first transmit aperture and the second transmit aperture at the same position in the long axis direction of the probe, to transmit the first transmission beam and the second transmission beam, respectively,
the receiver generates the first received beam signal and the second received beam signal for a reception scanning line on the same position, and
the signal synthesizer or the image synthesizer weights and synthesizes the first received beam signal and the second received beam signal on the same reception scanning line.

6. The ultrasound imaging apparatus according to claim 1, wherein
the transmitter sets the first transmit aperture while moving the position thereof in the long axis direction of the probe for each transmission by a predetermined amount and transmits the first transmission beams the number of which is required for generating the first frame data, and subsequently the transmitter sets the second transmit aperture while moving the position thereof in the long axis direction of the probe for each transmission by a predetermined amount and transmits the second transmission beams the number of which is required for generating the second frame data,
the receiver generates respectively the first received beam signals and the second received beam signals while moving the reception scanning line in the long axis direction of the probe, with the movement of the first transmit aperture and the second transmit aperture in the long axis direction of the probe,
the image former generates the first frame data from the first received beam signals, and generates the second frame data from the second received beam signals, and
the signal synthesizer or the image synthesizer weights and synthesizes the first frame data and the second frame data.

7. The ultrasound imaging apparatus according to claim 1, wherein
the probe has a switching unit configured to switch an aperture size of first transmit aperture and the second transmit aperture in the short-axis direction of the probe, and
the transmitter comprises a transmission control unit configured to switch the switching unit to the aperture size of the first transmit aperture and the second transmit aperture in the short axis direction of the probe.

8. The ultrasound imaging apparatus according to claim 1, wherein
the transmitter alternately sets the first transmit aperture and the second transmit aperture for each transmission while moving each position by a predetermined amount in the long axis direction of the probe,
the receiver moves positions of the reception scanning lines for forming the first received beam signal and the second received beam signal in the long axis direction of the probe, with the movement of the first transmit aperture and the second transmit aperture in the long axis direction, and
the synthesizer weights and synthesizes the first received beam signal and the second received beam signal respectively on the reception scanning lines being adjacent.

9. The ultrasound imaging apparatus according to claim 1, wherein
the transmitter alternately sets the first transmit aperture and the second transmit aperture for each transmission while moving each position by a predetermined amount in the long axis direction of the probe, transmits the first transmission beams and the second transmission beams the number of which is required for generating the first frame data, and subsequently after changing of positions between the first transmit aperture and the second transmit aperture, with respect to the positions thereof when generating the first frame data, the transmitter transmits the first transmission beams and the second transmission beams the number of which is required for generating the second frame data, and
the receiver generates the first received beam signals and the second received beam signals while moving the reception scanning lines in the long axis direction of probe, with the movement of the first transmit aperture and the second transmit aperture, respectively in the long axis direction of the probe,
the image former generates the first frame data from the first received beam signals, and generates the second frame data from the second received beam signals, and
the synthesizer weights and synthesizes the first received beam signals and the second received beam signals, or weights and synthesizes the first frame data and the second frame data.

10. The ultrasound imaging apparatus according to claim 1, wherein
when transmitting the transmission beam from the second transmit aperture, the transmitter does not output the transmission signal to the transducer in a central portion of the second transmit aperture in the short axis direction of the probe, so as not to transmit the second transmission beam from the transducer at the central portion.

11. The ultrasound imaging apparatus according to claim 1, wherein
the transmit aperture set by the transmitter, has three or more types of the aperture sizes in the short axis direction, for transmitting three or more types of transmission beams,
the receiver generates three or more types of received beam signals respectively corresponding to the three or more transmission beams, and
the synthesizer sets a weight assigned by the weighting, in such a manner that at a deeper position in the subject, the received beam signal or the frame data obtained by setting the transmit aperture having a larger aperture size in the short axis direction of the probe is weighted larger, in at least a portion of the depth region.

12. The ultrasound imaging apparatus according to claim 1, wherein
while the transmitter repeats the operation of setting the first transmit aperture with moving the position thereof for each transmission by a predetermined amount in the long axis direction of probe, transmitting the first transmission beams the number of which is required for generating the first frame data, thereafter, while the transmitter repeats the operation of setting the second transmit aperture with moving the position thereof for each transmission by a predetermined amount in the long axis direction of the probe, transmitting the second transmission beams the number of which is required for generating the second frame data; the transmitter sequentially switches each of irradiation angles of the first transmission beam and the second transmission beam with respect to the depth direction of the subject to predetermined multiple types of angles, each time a frame number is incremented,
the image former generates frame data for each of the frame numbers, and
the signal synthesizer or the image synthesizer weights and synthesizes the frame data the number of which is equal to or larger than the number of the types of the irradiation angles.

13. The ultrasound imaging apparatus according to claim 12, wherein
the transmitter sets the first transmit aperture or the second transmit aperture being predetermined for each of predetermined multiple types of irradiation angles.

14. The ultrasound imaging apparatus according to claim 12, wherein
when synthesizing the frame data the number of which is equal to or larger than the number of the irradiation angle types, the signal synthesizer weights and synthesizes the received beam signals respectively corresponding to the position in the long axis direction of the probe, among the received beam signals constituting the frame data the number of which is equal to or larger than the number of the multiple types of irradiation angles.

15. The ultrasound imaging apparatus according to claim 1, wherein
while the transmitter repeats the operation of setting alternately the first transmit aperture and the second transmit aperture for each transmission, with moving the position thereof by a predetermined amount in the long axis direction of probe, transmits the first transmission beams and the second transmission beams the number of which is required for generating the first frame data, and subsequently after changing of positions between the first transmit aperture and the second transmit aperture, with respect to the positions thereof when generating the first frame data, transmitting the first transmission beams and the second transmission beams the number of which is required for generating the second frame data; the transmitter sequentially switches each of the irradiation angle of the first transmission beam and the second transmission beam with respect to the depth direction of the subject to predetermined multiple types of angles, each time a frame number is incremented,
the receiver generates the first received beam signal and the second received beam signal while moving the reception scanning lines in the long axis direction of the probe, with the movement of the first transmit aperture and the second transmit aperture respectively in the long axis direction of the probe,
the signal synthesizer performs weighting and synthesizing between the first received beam signals and the second received beam signals respectively at the same positions in the long axis direction of the probe, among the first received beam signals and the second received signals generated by the receiver based on the first transmission beams and the second transmission beams for generating the first frame data, and the first received beam signals and the second received signals generated by the receiver based on the first transmission beams and the second transmission beams for generating the second frame data,
the image former generate frame data using received beam signals after synthesized by the signal synthesizer, and
the image synthesizer synthesizes the frame data the number of which is equal to or more than the number of types of the irradiation angles.

16. An ultrasound imaging method, comprising
setting sequentially a first transmit aperture having a predetermined aperture size in a short axis direction of a probe and a second transmit aperture having the aperture size in the short axis direction of the probe larger than the first transmit aperture, in the probe where transducers are arranged in each of a long axis direction and the short axis direction, and outputting transmission signals to the transducers in each of the first transmit aperture and the second transmit aperture, thereby transmitting a first transmission beam and a second transmission beam sequentially, to a subject from the transducers,
receiving the received signals that the transducers of the probe receive reflected waves of the first transmission beam and the second transmission beam respectively from the subject and output, and beamforming received signals in the long axis direction of the probe to generate a first received beam signal and a second received beam signal,
generating frame data using the first received beam signal and the second received beam signal, and
weighting and synthesizing the first received beam signal and the second received beam signal, or weighting and synthesizing s first frame data generated from the first received beam signal and a second frame data generated from the second received beam signal.
